(19) United States
(12) Patent Application Publication
(10) Pub. No.: US 2022/0273575 A1
(43) Pub. Date: Sep. 1, 2022

(54) PARTICLES, PHARMACEUTICAL COMPOSITION, AND METHOD FOR PRODUCING PARTICLES

(71) Applicant: RICOH COMPANY, LTD., Tokyo (JP)

(72) Inventors: Tatsuru MORITANI, Kanagawa (JP); Nana Akiba, Kanagawa (JP); Tadahiko Morinaga, Kanagawa (JP)

(73) Assignee: Ricoh Company, Ltd., Tokyo (JP)

(21) Appl. No.: 17/597,604

(22) PCT Filed: Aug. 7, 2020
(86) PCT No.: PCT/JP2020/030923

§ 371 Date: Jan. 13, 2022

(30) Foreign Application Priority Data
Aug. 9, 2019 (JP) 2019-147795

(51) Int. Cl. A61K 9/16; A61K 47/38

(52) U.S. Cl. CPC A61K 9/1682; A61K 9/1652; A61K 9/1647; A61K 47/38

(57) ABSTRACT

Particles including at least one base material and a physiologically active substance having physiological activity are useful. The physiologically active substance is contained and dispersed in the at least one base material, and the physiologically active substance has a property such that the physiological activity is changed by heating, cooling, or external stress.

### TECHNICAL FIELD

The present invention relates to particles, a pharmaceutical composition, and a method for producing particles. Priority is claimed on Japanese Patent Application No. 2019-147795, filed Aug. 9, 2019, the content of which is incorporated herein by reference.

### BACKGROUND ART

In recent years, research has been actively performed on a drug delivery system as a technology for efficiently and safely administering medicinal components to disease sites. Among these, there is an increased demand for fine particles which have a particle diameter of, for example, less than or equal to several hundred nm and contain or carry medicinal components in order to deliver the medicinal components into blood vessels.

Examples of a method for producing fine particles in which physiologically active substances such as medicinal components are dispersed include an emulsion solvent diffusion method (ESD method) or a spray-drying method. The ESD method is a method for producing the above-described fine particles by stirring a liquid containing an organic polymer, a physiologically active substance, and a good solvent while diffusing the liquid in a poor solvent. In addition, the spray-drying method is a method for producing the above-described fine particles by spraying a liquid containing an organic polymer and a physiologically active substance, and heating and drying the liquid.

For example, powder obtained by spray-drying a water-soluble substance selected from peptides, proteins, glycoproteins, saccharides, and nucleic acids is disclosed in Patent Document 1.

### SUMMARY OF INVENTION

### Technical Problem

However, in a case of using the method for producing particles known in the related art, in many cases, the physiological activity of a physiologically active substance contained in a base material constituting particles changes or is deactivated by heating, cooling, shaking, stirring, and the like during a production step, or the physiologically active substance is eluted by a solvent or the like during the production step, and therefore does not come to be contained in the particles. As a result, there is a problem in that the amount of desired physiological activity for the produced particles decreases.

### Solution to Problem

The present invention relates to particles, including: at least one base material; and a physiologically active substance having physiological activity, in which the physiologically active substance is contained and dispersed in the at least one base material, and the physiologically active substance has a property such that the physiological activity is changed by heating, cooling, or external stress.

### Advantageous Effects of Invention

The particles of the present invention exhibit an excellent effect of suppressing a reduction in the amount of physiological activity of the particles in which a physiologically active substance having a property such that the physiological activity is changed by heating, cooling, or external stress is contained and dispersed in a base material.

### BRIEF DESCRIPTION OF DRAWINGS

FIG. 1 is a schematic cross-sectional view showing an example of liquid column resonance liquid droplet discharge means.

FIG. 2 is a schematic diagram showing an example of a particle production device.

FIG. 3 is a schematic diagram showing another example of a particle production device.

FIG. 4A is a schematic diagram showing an example of a particle production device capable of providing a flow of a poor solvent to a discharge unit of liquid droplet discharge means.

FIG. 4B is an enlarged view of the vicinity (broken line portion) of the liquid droplet discharge means in FIG. 4A.

FIG. 5 is a schematic diagram showing an example of a particle production device having good solvent removal means that removes a good solvent.

FIG. 6 is a view showing an example of a particle size distribution of particles produced through a method of a second embodiment and particles produced through a spray-drying method.

FIG. 7 is a schematic diagram showing an example of a particle production device.

FIG. 8 is a schematic cross-sectional view showing an example of liquid droplet discharge means used in the particle production device.

FIG. 9 is a schematic cross-sectional view showing another example of liquid droplet discharge means used in a particle production device.

### DESCRIPTION OF EMBODIMENTS

Hereinafter, the present invention will be described in detail.

### <<Particles>>

Particles of the present invention contain at least one base material and a physiologically active substance having physiological activity, and contain other materials as necessary. Any physiologically active substance may be used as long as it has some physiological activity in a living body. As a preferred aspect, the physiologically active substance has a property such that physiological activity is changed by physical or chemical stimulation such as heating, cooling, shaking, stirring, and pH change.

“Particles” mentioned in the present application mean a group of granular compositions containing a base material and a physiologically active substance, unless otherwise specified. The particles of the present invention are typically functional particles exhibiting a desired function. The particles of the present invention can be designed so as to become functional particles having a desired function by appropriately selecting a base material to be contained therein. Examples of functional particles include particles that deliver a physiologically active substance to a target site in order to exhibit a desired physiological effect, that is, particles used in a drug delivery system (DDS), or sustained-release particles that continuously release a drug over a longer period of time, or solubilization particles for solubilizing a poorly soluble physiologically active substance.

A “base material” in the present application is a component contained in particles, and is a material serving as a base constituting each particle.

A “physiologically active substance” in the present application is an active component used for exhibiting a physiological effect in a living body, and examples thereof include polymer compounds including biopolymers, such as proteins such as antibodies and enzymes and nucleic acids such as DNA and RNA, in addition to low-molecular-weight compounds including pharmaceutical compounds, food compounds, and cosmetic compounds. In addition, the “physiological effect” is an effect produced by a physiologically active substance exhibiting physiological activity at a target site, and has quantitative and/or qualitative changes and effects on, for example, a living body, tissues, cells, proteins, DNA, and RNA. In addition, the term “physiological activity” means that a physiologically active substance acts on a target site (for example, a target tissue) to change and affect it. A receptor or the like existing on the surface of a cell or inside a cell is preferable as a target site, for example. In this case, a signal is transmitted to cells by the physiological activity of a physiologically active substance binding to a specific receptor, and as a result, a physiological effect is exhibited. The physiologically active substance may be a substance which is converted into a mature form by enzymes in a living body, and then binds to a specific receptor to exhibit a physiological effect. In this case, a substance before being converted into a mature form is also regarded as being included in the physiologically active substance in the present application. The physiologically active substance may be a substance produced by an organism (human or non-human organism), or may be a substance artificially synthesized.

Examples of “properties of changing physiological activity” in the present application include a property of increasing or decreasing the amount of physiological activity, a property of increasing or decreasing the efficiency of physiological activity, and a property of changing the type of physiological activity, but is preferably a property of decreasing the amount of physiological activity or a property of decreasing the efficiency of physiological activity, and more preferably a property of decreasing the amount of physiological activity. In addition, examples of changes in physiological activity include reversible changes and irreversible changes, but a property of irreversibly changing physiological activity is preferable.

“Heating” and “cooling” in the present application is typically applying heat energy to a liquid containing a physiologically active substance and taking heat energy from the liquid. In some cases, “heating” or “cooling” may change physiological activity due to changes in molecular structure or three-dimensional structure of a physiologically active substance or the like. Specific examples thereof include thermal denaturation of proteins and low-temperature denaturation of proteins in a case where a physiologically active substance is a protein. In addition, examples thereof include decomposition of nucleic acids in a case where a physiologically active substance is a nucleic acid. As described above, a “temperature at which the physiological activity of a physiologically active substance changes” varies depending on the type of physiologically active substance to be selected. However, those skilled in the art will be able to easily recognize such temperatures.

An “external stress” in the present application is typically a force applied to a liquid containing a physiologically active substance from the outside. Examples of the external stresses include shaking, stirring, and shear stress. In some cases, application of these kinds of external stresses may change the physiological activity due to changes in molecular structure or three-dimensional structure of a physiologically active substance or the like. Specific examples thereof include deactivation of proteins due to changes in higher-order structure or the like in a case where a physiologically active substance is a protein. Examples of proteins that are easily deactivated by external stress include proteins forming a multimer, and specific examples thereof include enzymes and antibodies. Examples of treatments generating external stress include a shaking treatment, an agitation treatment, a pulverization treatment, an ultrasound treatment, a homogenizer treatment, and a spray treatment. Although whether or not external stress generated by these treatments corresponds to the “external stress by which the physiological activity of the physiologically active substance changes” varies depending on the type of physiologically active substance to be selected, those skilled in the art will be able to easily recognize such external stresses.

### <Form of Particles>

Next, the form of particles will be described. In general, examples of forms of DDS particles containing a base material and a physiologically active substance include capsule particles which are in a form in which a physiologically active substance is encapsulated in a base material, carrier particles in which a physiologically active substance is carried on the surface of a base material, and particles in other forms.

Examples of capsule particles include dispersion encapsulant particles which are in a form in which a physiologically active substance is dispersed and encapsulated in a base material, and uneven distribution encapsulant particles which are in a form in which a physiologically active substance is unevenly distributed and encapsulated in a base material. “Encapsulation” in the capsule particles is not particularly limited as long as a physiologically active substance is temporarily or continuously held in a base material.

The dispersion encapsulant particles are not particularly limited as long as a physiologically active substance is dispersed and encapsulated in a base material, and the degree of dispersion of a physiologically active substance may not be uniform. In addition, in a case where particles contain plural kinds of base materials and one of the base materials is unevenly distributed at a predetermined site in the particles, the degree of dispersion may vary depending on the types of base materials at sites where a physiologically active substance is encapsulated. Examples of particles corresponding to dispersion encapsulant particles include the particles of the present invention, particles produced through an emulsion solvent diffusion method (ESD method), and particles produced through a spray-drying method.

The uneven distribution encapsulant particles are in a form in which a physiologically active substance is unevenly distributed and encapsulated in a base material, and in other words, are in a form in which a physiologically active substance is encapsulated in the base material when the base material and the physiologically active substance in the particles are positioned substantially separated from each other. Examples of forms of uneven distribution encapsulant particles include a form of particles having a central portion containing a physiologically active substance and an outer circumferential portion which contains a base material and includes the central portion. Examples of particles corresponding to uneven distribution encapsulant particle include liposomes, micelles, and coated particles.

Carrier particles are in a form in which a physiologically active substance is carried by being adsorbed or bound to the surface of a base material. Examples of the type of adsorption include chemical adsorption and physical adsorption. Examples of the type of binding include hydrogen bonding, covalent bonding, ionic bonding, and chelate bonding. Examples of particles corresponding to carrier particles include porous particles in which a physiologically active substance is carried on the surface (including not only the outer surface but also the inner surface) of a porous base material.

The particles of the present invention contain a physiologically active substance dispersed in at least one base material, and are classified as capsule particles, further classified as dispersion encapsulant particles, and particularly classified as solid dispersion particles to be described below.

In addition, the particles of the present invention may contain two or more base materials. In the case where the particles of the present invention contain two or more base materials, the particles may be in a form in which one of the base materials is unevenly distributed on a surface side of a particle. In this case, a physiologically active substance can be dispersed and encapsulated in both a base material (hereinafter, also referred to as a “surface base material”) unevenly distributed on a surface side of a particle and a base material (hereinafter, also referred to as an “inner base material”) other than the surface base material. In addition, specific examples of this form include a form in which a physiologically active substance is unevenly distributed on the surface base material side and a form in which a physiologically active substance is unevenly distributed on an inner base material side, but a form in which a physiologically active substance is unevenly distributed on an inner base material side is preferable. In a case where a physiologically active substance is unevenly distributed on the inner base material side, sustained-release particles in which the elution rate of a physiologically active substance is suppressed can be produced.

A method for confirming that the particles contain at least two base materials and are in a form in which one of the at least two base materials is unevenly distributed on a surface side of a particle is not particularly limited and can be appropriately selected depending on the purpose. Examples of the method for confirming thereof include a method of observing cross sections of particles with a scanning electron microscope, a transmission electron microscope, a scanning probe microscope, or the like. In addition, examples of other methods for confirming thereof include a method of confirming that particles are the above-described particles when it is possible to determine that a component of a surface base material which has been measured through a time-of-flight secondary ion mass method is different from a component of an inner base material. Furthermore, as the other methods for confirming thereof, it is possible to perform pretreatment such as electronic dyeing or a dissolution treatment. For examples, in a case where the above-described particles consist of a base material of a water-soluble component and a base material of a water-insoluble component, it may be determined that particles are the above-described particles by immersing cross sections of the particles and observing the cross sections on which the water-soluble component completely dissolves using a scanning electron microscope to determine that the water-insoluble component has been distributed in a remaining portion of the cross sections of the particles and the water-soluble component has been distributed in a void portion.

In addition, the particles of the present invention are preferably a solid dispersion. A solid dispersion means that a physiologically active substance is dispersed in particles in an amorphous state. In a case where a physiologically active substance is held in an amorphous state, it is in a higher energy state compared to a crystalline state, and the solubility of the physiologically active substance is improved.

In general, in a case where a physiologically active substance is used in a state of being dissolved in a liquid, there is an advantage that adjustment work immediately before use can be omitted by storing the physiologically active substance in this state, but there is a disadvantage that a storable time may be short. On the other hand, in a case where a physiologically active substance is stored in a solid state is used, it is necessary to dissolve the physiologically active substance in a liquid immediately before use. Therefore, there is a disadvantage that the time required for the dissolution may be long, but there is an advantage that a preservable period is extended. In this respect, in the case where a physiologically active substance preserved in a solid dispersion state is used, it is necessary to dissolve the physiologically active substance in a liquid immediately before use. However, since the physiologically active substance has high solubility, there are advantages that the time required for dissolution is shortened and the preservable period is extended, which is preferable.

In addition, in a case where a physiologically active substance is a poorly water-soluble substance and is used through oral administration or the like, there is a problem that the physiologically active substance has low bioavailability. In such a case, the solubility and the bioavailability of the physiologically active substance can be improved by making the particles of the present invention into a solid dispersion. A poorly water-soluble substance means a substance of which a log P value of a water-octanol distribution coefficient is greater than or equal to 3, and a water-soluble substance means a compound of which a log P value of a water-octanol distribution coefficient is less than 3. The water-octanol distribution coefficient can be measured according to a flask-shaking method of JIS Z 7260-107 (2000).

### <Materials Constituting Particles>

Materials constituting the particles include a base material and a physiologically active substance.

### —Base Material—

A base material is a material serving as a base constituting particles. Accordingly, the base material is preferably a solid at normal temperature. The base material is not particularly limited as long as it is not a substance adversely affecting a physiologically active substance also contained in the particles, and may be a low-molecular-weight substance or a high-molecular-weight substance. Since the particles of the present invention are preferably particles applicable to living bodies, the base material is preferably a substrate which is not toxic to living bodies. The low-molecular-weight substance is preferably a compound having a weight-average molecular weight of less than 15,000. The high-molecular-weight substance is preferably a compound having a weight-average molecular weight of greater than or equal to 15,000. As described above, a base material may be used alone or in combination of two or more thereof, and any base material to be described below may be used in combination.

### —Low-Molecular-Weight Substance—

The low-molecular-weight substance is not particularly limited, and can be appropriately selected depending on the purpose. Examples thereof include lipids, saccharides, cyclodextrins, amino acids, and organic acids. These may be used alone or in combination of two or more thereof.

### —Lipids—

The lipids are not particularly limited, and can be appropriately selected depending on the purpose. Examples thereof include medium-chain or long-chain monoglycerides, medium-chain or long-chain diglycerides, medium-chain or long-chain triglycerides, phospholipids, vegetable oils (for example, soybean oil, avocado oil, squalene oil, sesame oil, olive oil, corn oil, rapeseed oil, safflower oil, and sunflower oil), fish oil, seasoning oil, water-insoluble vitamins, fatty acids, mixtures thereof, and derivatives thereof. These may be used alone or in combination of two or more thereof.

### —Saccharides—

The saccharides are not particularly limited, and can be appropriately selected depending on the purpose. Examples thereof include monosaccharides or polysaccharides such as glucose, mannose, idose, galactose, fucose, ribose, xylose, lactose, sucrose, maltose, trehalose, turanose, raffinose, maltotriose, acarbose, cyclodextrins, amylose (starch), and cellulose; sugar alcohols (polyols) such as glycerin, sorbitol, lactitol, maltitol, mannitol, xylitol, and erythritol; and derivatives thereof. These may be used alone or in combination of two or more thereof.

### —Cyclodextrins—

The cyclodextrins are not particularly limited, and can be appropriately selected depending on the purpose. Examples thereof include hydroxypropyl-β-cyclodextrin, β-cyclodextrin, γ-cyclodextrin, α-cyclodextrin, and cyclodextrin derivatives. These may be used alone or in combination of two or more thereof.

### —Amino Acids—

The amino acids are not particularly limited, and can be appropriately selected depending on the purpose. Examples thereof include valine, lysine, leucine, threonine, isoleucine, asparagine, glutamine, phenylalanine, aspartic acid, serine, glutamic acid, methionine, arginine, glycine, alanine, tyrosine, proline, histidine, cysteine, tryptophan, and derivatives thereof. These may be used alone or in combination of two or more thereof.

### —Organic Acids—

The organic acids are not particularly limited, and can be appropriately selected depending on the purpose. Examples thereof include adipic acid, ascorbic acid, citric acid, fumaric acid, gallic acid, glutaric acid, lactic acid, malic acid, maleic acid, succinic acid, tartaric acid, and derivatives thereof. These may be used alone or in combination of two or more thereof.

### —High-Molecular-Weight Substance—

The high-molecular-weight substance is not particularly limited, and can be appropriately selected depending on the purpose. Examples thereof include proteins and polysaccharides such as water-soluble celluloses, polyalkylene glycols, poly(meth)acrylamides, poly(meth)acrylic acids, poly(meth)acrylic acid esters, polyallylamines, polyvinyl pyrrolidone, polyvinyl alcohols, polyvinyl acetates, biodegradable polyesters, polyglycolic acid, polyamino acids, gelatin, and fibrin, and derivatives thereof.

These may be used alone or in combination of two or more thereof.

### —Water-Soluble Celluloses—

The water-soluble celluloses are not particularly limited, and can be appropriately selected depending on the purpose. Examples thereof include alkyl celluloses such as methyl cellulose and ethyl cellulose; hydroxyalkyl celluloses such as hydroxyethyl cellulose and hydroxypropyl cellulose; and hydroxyalkyl alkyl celluloses such as hydroxyethyl methyl cellulose and hydroxypropyl methyl cellulose. These may be used alone or in combination of two or more thereof. Among these, hydroxypropyl cellulose and hydroxypropyl methylcellulose are preferable and hydroxypropyl cellulose is more preferable from the viewpoints of high biocompatibility and high solubility in a solvent used when producing particles.

### —Hydroxypropyl Cellulose—

As hydroxypropyl cellulose, various products having different viscosities are commercially available from various companies, and all of them can be used in the base material of the present invention. The viscosity of a 2 mass % aqueous solution of hydroxypropyl cellulose (at 20° C.) is not particularly limited, and can be appropriately selected depending on the purpose, but is preferably 2.0 mPa·s (centipoise, cps) to 4,000 mPa·s (centipoise, cps).

In addition, it is conceivable that the viscosity of hydroxypropyl cellulose may depend on the weight-average molecular weight, the degree of substitution, and the molecular weight of the hydroxypropyl cellulose. The weight-average molecular weight of hydroxypropyl cellulose is not particularly limited, and can be appropriately selected depending on the purpose, but is preferably 15,000 to 400,000. The-weight average molecular weight can be measured, for example, through gel permeation chromatography (GPC).

Commercially available products of hydroxypropyl cellulose are not particularly limited, and can be appropriately selected depending on the purpose. Examples thereof include HPC-SSL having a molecular weight of 15,000 to 30,000 and a viscosity of 2.0 mPa·s to 2.9 mPa·s, HPC-SL having a molecular weight of 30,000 to 50,000 and a viscosity of 3.0 mPa·s to 5.9 mPa·s, HPC-L having a molecular weight of 55,000 to 70,000 and a viscosity of 6.0 mPa·s to 10.0 mPa·s, HPC-M having a molecular weight of 110,000 to 150,000 and a viscosity of 150 mPa·s to 400 mPa·s, and HPC-H having a molecular weight of 250,000 to 400,000 and a viscosity of 1,000 mPa·s to 4,000 mPa·s (all are manufactured by NIPPON SODA CO., LTD.) These may be used alone or in combination of two or more thereof. Among these, HPC-SSL having a molecular weight of 15,000 to 30,000 and a viscosity of 2.0 mPa·s to 2.9 mPa·s is preferable. In the above-described commercially available products, the molecular weight is measured through gel permeation chromatography (GPC) and the viscosity is measured using a 2 mass % aqueous solution (at 20° C.).

The content of hydroxypropyl cellulose is not particularly limited, and can be appropriately selected depending on the purpose, but is, based on the mass of a base material, preferably greater than or equal to 50 mass %, more preferably 50 mass % to 99 mass %, still more preferably 75 mass % to 99 mass %, and particularly preferably 80 mass % to 99 mass %.

### —Polyalkylene Glycols—

The polyalkylene glycols are not particularly limited, and can be appropriately selected depending on the purpose. Examples thereof include polyethylene glycol (PEG), polypropylene glycol, polybutylene glycol, and copolymers thereof. These may be used alone or in combination of two or more thereof.

### —Poly(Meth)Acrylamides—

The poly(meth)acrylamides are not particularly limited, and can be appropriately selected depending on the purpose. Examples thereof include polymers of monomers such as N-methyl(meth)acrylamide, N-ethyl(meth)acrylamide, N-propyl(meth)acrylamide, N-butyl(meth)acrylamide, N-benzyl(meth)acrylamide, N-hydroxyethyl(meth)acrylamide, N-phenyl(meth)acrylamide, N-tolyl(meth)acrylamide, N-(hydroxyphenylxmeth)acrylamide, N-(sulfamoylphenyl)(meth)acrylamide, N-(phenylsulfonylxmeth)acrylamide, N-(tolylsulfonyl)(meth)acrylamide, N,N-dimethyl(meth)acrylamide, N-methyl-N-phenyl (meth)acrylamide, and N-hydroxyethyl-N-methyl(meth)acrylamide. These monomers may be polymerized alone or in combination of two or more thereof. In addition, these polymers may be used alone or in combination of two or more thereof.

### —Poly(Meth)Acrylic Acids—

The poly(meth)acrylic acids are not particularly limited, and can be appropriately selected depending on the purpose. Examples thereof include homopolymers such as polyacrylic acid and polymethacrylic acid, and copolymers such as an acrylic acid-methacrylic acid copolymer. These may be used alone or in combination of two or more thereof.

### —Poly(Meth)Acrylic Acid Esters—

The poly(meth)acrylic acid esters are not particularly limited, and can be appropriately selected depending on the purpose. Examples thereof include polymers of monomers such as ethylene glycol di(meth)acrylate, diethylene glycol di(meth)acrylate, propylene glycol di(meth)acrylate, glycerol poly(meth)acrylate, polyethylene glycol (meth)acrylate, trimethylolpropane tri(meth)acrylate, pentaerythritol tetra(meth)acrylate, and 1,3-butylene glycol di(meth)acrylate. These monomers may be polymerized alone or in combination of two or more thereof. In addition, these polymers may be used alone or in combination of two or more thereof.

### —Polyallylamines—

The polyallylamines are not particularly limited, and can be appropriately selected depending on the purpose. Examples thereof include diallylamine and triallylamine. These may be used alone or in combination of two or more thereof.

### —Polyvinyl Pyrrolidone—

Commercially available products can be used as the polyvinyl pyrrolidone. Commercially available products of polyvinyl pyrrolidone are not particularly limited, and can be appropriately selected depending on the purpose. Examples thereof include Plasdone C-15 (ISP TECHNOLOGIES), Kolidon VA64, Kolidon K-30, Kolidon CL-M (all are manufactured by KAWARLAL), and Kollicoat IR (manufactured by BASF). These may be used alone or in combination of two or more thereof.

### —Polyvinyl Alcohols—

The polyvinyl alcohols are not particularly limited, and can be appropriately selected depending on the purpose. Examples thereof include silanol-modified polyvinyl alcohol, carboxyl-modified polyvinyl alcohol, and acetoacetyl-modified polyvinyl alcohol. These may be used alone or in combination of two or more thereof.

### —Polyvinyl Acetates—

The polyvinyl acetates are not particularly limited, and can be appropriately selected depending on the purpose. Examples thereof include a vinyl acetate-crotonic acid copolymer and a vinyl acetate-itaconic acid copolymer. These may be used alone or in combination of two or more thereof.

### —Biodegradable Polyesters—

The biodegradable polyesters are not particularly limited, and can be appropriately selected depending on the purpose. Examples thereof include polylactic acid; poly-ε-caprolactone; succinate polymers such as polyethylene succinate, polybutylene succinate and polybutylene succinate adipate; polyhydroxyalkanoates such as polyhydroxypropionates, polyhydroxybutyrates, and polyhydroxyvalerates; and polyglycolic acid. These may be used alone or in combination of two or more thereof. Among these, polylactic acid is preferable from the viewpoint of high biocompatibility and being capable of eluting a contained physiologically active substance in a sustained-release manner.

### —Polylactic Acid—

The weight-average molecular weight of the polylactic acid is not particularly limited, and can be appropriately selected depending on the purpose, but is preferably 5,000 to 100,000, more preferably 10,000 to 70,000, still more preferably 10,000 to 50,000, and particularly preferably 10,000 to 30,000.

The content of polylactic acid is not particularly limited, and can be appropriately selected depending on the purpose, but is, based on the mass of a base material, preferably greater than or equal to 50 mass %, more preferably 50 mass % to 99 mass %, still more preferably 75 mass % to 99 mass %, and particularly preferably 80 mass % to 99 mass %.

### —Polyglycolic Acids—

The polyglycolic acids are not particularly limited, and can be appropriately selected depending on the purpose. Examples thereof include a lactic acid-glycolic acid copolymer which is a copolymer having a structural unit derived from lactic acid and a structural unit derived from glycolic acid, a glycolic acid-caprolactone copolymer which is a copolymer having a structural unit derived from glycolic acid and a structural unit derived from caprolactone, and a glycolic acid-trimethylene carbonate copolymer which is a copolymer having a structural unit derived from glycolic acid and a structural unit derived from trimethylene carbonate. These may be used alone or in combination of two or more thereof. Among these, a lactic acid-glycolic acid copolymer is preferable from the viewpoints of high biocompatibility, being capable of eluting a contained physiologically active substance in a sustained-release manner, and preserving a contained physiologically active substance for a long period of time.

The weight-average molecular weight of a lactic acid-glycolic acid copolymer is not particularly limited, and can be appropriately selected depending on the purpose, but is preferably 2,000 to 250,000, more preferably 2,000 to 100,000, still more preferably 3,000 to 50,000, and particularly preferably 5,000 to 10,000.

The molar ratio (L:G) between a structural unit (L) derived from lactic acid and a structural unit (G) derived from glycolic acid in a lactic acid-glycolic acid copolymer is not particularly limited, and can be appropriately selected depending on the purpose, but is preferably 1:99 to 99:1, more preferably 25:75 to 99:1, still more preferably 30:70 to 90:10, and particularly preferably 50:50 to 85:15.

The content of lactic acid-glycolic acid copolymer is not particularly limited, and can be appropriately selected depending on the purpose, but is, based on the mass of a base material, preferably greater than or equal to 50 mass %, more preferably 50 mass % to 99 mass %, still more preferably 75 mass % to 99 mass %, and particularly preferably 80 mass % to 99 mass %.

### —Polyamino Acids—

The polyamino acids are not particularly limited, and can be appropriately selected depending on the purpose. Polyamino acids may be obtained by arbitrarily combining amino acids exemplified in the section of the above-described amino acids and polymerizing the combined amino acids, but are preferably obtained by polymerizing a single amino acid. Examples of preferred polyamino acids include amino acid homopolymers such as poly-α-glutamic acid, poly-γ-glutamic acid, polyaspartic acid, polylysine, polyarginine, polyornithine, and polyserine, and copolymers thereof. These may be used alone or in combination of two or more thereof.

### —Gelatin—

Gelatin is not particularly limited, and can be appropriately selected depending on the purpose. Examples thereof include lime-treated gelatin, acid-treated gelatin, gelatin hydrolysate, and a gelatin enzyme dispersion, and derivatives thereof. These may be used alone or in combination of two or more thereof.

A natural dispersant polymer used in a gelatin derivative is not particularly limited, and can be appropriately selected depending on the purpose. Examples thereof include proteins, polysaccharides, and nucleic acids. A copolymer consisting of a natural dispersant polymer or a synthetic dispersant polymer is also included therein. These may be used alone or in combination of two or more thereof.

A gelatin derivative means gelatin derivatized via covalent bonding of a hydrophobic group to a gelatin molecule. A hydrophobic group is not particularly limited, and can be appropriately selected depending on the purpose. Examples thereof include polyesters such as polylactic acid, polyglycolic acid, and poly-s-caprolactone; lipids such as cholesterol and phosphatidylethanolamine; alkyl groups, aromatic groups containing a benzene ring; and heteroaromatic groups, and mixtures thereof.

The proteins are not particularly limited as long as these do not adversely affect the physiological activity of a physiologically active substance, and can be appropriately selected depending on the purpose. Examples thereof include collagen, fibrin, and albumin. These may be used alone or in combination of two or more thereof.

The polysaccharides are not particularly limited, and can be appropriately selected depending on the purpose. Examples thereof include chitin, chitosan, hyaluronic acid, alginic acid, starch, and pectin. These may be used alone or in combination of two or more thereof.

### —In Case of Containing at Least Two Base Materials—

In the case where the particles of the present invention contain at least two base materials and are in a form in which one of the at least two base materials is unevenly distributed on a surface side of a particle, the base materials are not particularly limited, and can be appropriately selected depending on the purpose. However, it is preferable that at least one base material have pH responsiveness, and it is more preferable that a base material unevenly distributed on a surface side of a particle have pH responsiveness.

The pH responsiveness means that solubility changes in response to pH. As an example of pH responsiveness, a base material dissolves at a pH of greater than or equal to 5.0. In this case, enteric particles can be produced by unevenly incorporating a base material, which has pH responsiveness and dissolves at a pH of greater than or equal to 5.0, on a surface side of a particle.

The base material having pH responsiveness is not particularly limited, and can be appropriately selected depending on the purpose. Examples thereof include a cellulosic polymer, a methacrylic acid-based polymer, a vinyl polymer, an amino acid, chitosan, pectin, and alginic acid. These may be used alone or in combination of two or more thereof. Among these, in a case where a base material having pH responsiveness is at least one selected from a cellulosic polymer and a methacrylic acid-based polymer, it is easier to unevenly incorporate the base material on a surface side of a particle during production of particles and to improve enteric properties compared with other base materials having pH responsiveness, which is preferable.

### —Cellulosic Polymers—

Examples of cellulosic polymers include hydroxypropyl methylcellulose acetate succinate, hydroxypropyl methylcellulose phthalate, carboxymethylethylcellulose, and cellulose acetate trimellitate. These may be used alone or in combination of two or more thereof. Among these, in a case where a cellulosic polymer is at least one selected from a hydroxypropyl methylcellulose acetate succinate and hydroxypropyl methylcellulose phthalate, it is easier to unevenly incorporate the base material on a surface side of a particle during production of particles and to improve enteric properties compared with other base materials having pH responsiveness, which is preferable.

### —Methacrylic Acid-Based Polymers—

Examples of methacrylic acid-based polymers include an aminoalkyl methacrylic ester copolymer, a methacrylic acid copolymer, a methacrylic ester copolymer, and an ammonioalkyl methacrylic ester copolymer. These may be used alone or in combination of two or more thereof. Among these, in a case where a methacrylic acid-based polymer is an ammonioalkyl methacrylic ester copolymer, it is easier to unevenly incorporate the base material on a surface side of a particle during production of particles and to improve enteric properties compared with other base materials having pH responsiveness, which is preferable.

The combination of at least two base materials is not particularly limited, and can be appropriately selected depending on the purpose. For example, in a case where there are two base materials, a combination of any one selected from poly(meth)acrylic acids, polyglycolic acid, and hydroxypropyl methylcellulose and any one selected from hydroxypropyl cellulose, polyethylene pyrrolidone, and polyalkylene glycols is preferable.

A base material is preferably a substance capable of incorporating particles containing the base material in pharmaceutical preparations, functional foods, functional cosmetics, and the like. Therefore, among the above-described materials, a substance having no biotoxicity, particularly a biodegradable substance such as a biodegradable polymer is preferable.

The content of base material with respect to the mass of particles is preferably 5 mass % to 95 mass % and more preferably 50 mass % to 95 mass %. In a case where the content of base material is 5 mass % to 95 mass %, redispersibility of a physiologically active substance in water improves due to an action of the base material.

### —Physiologically Active Substance—

The physiologically active substance is an active component used for causing a living body to exhibit a physiological effect. In addition, the physiologically active substance has a property such that the physiological activity is changed by at least one selected from heating, cooling, or shaking. The physiological activity may be changed by heating and cooling, but may not be changed by shaking. The physiological activity may be changed by heating and shaking, but may not be changed by cooling. The physiological activity may be changed by cooling and shaking, but may not be changed by heating. The physiological activity may be changed by heating, cooling, and shaking.

Examples of physiologically active substances include a physiologically active substance contained in pharmaceutical compositions, a physiologically active substance contained in functional foods, and a physiologically active substance contained in functional cosmetics. These may be used alone or in combination of two or more thereof.

### —Physiologically Active Substances Contained in Pharmaceutical Composition—

Physiologically active substances contained in pharmaceutical compositions are not particularly limited, and can be appropriately selected depending on the purpose. Examples thereof include nucleic acids, polypeptides containing proteins, carbohydrates, lipids, and low-molecular-weight compounds. These may be used alone or in combination of two or more thereof.

### —Nucleic Acids—

Examples of nucleic acids typically include DNA, RNA, and a combination thereof. Nucleic acids may be replaced with chemically modified nucleic acids obtained by a part or all of these sequences being chemically modified. In addition, chemically synthesized nucleic acid analogs such as peptide nucleic acid (PNA) and morpholino antisense oligo are also included in nucleic acids. In addition, in a case where it is aimed to suppress expression of a target gene, examples of nucleic acids include antisense nucleic acids against a transcription product of a target gene or a part thereof, nucleic acids having ribozyme activity of specifically cleaving a transcription product of a target gene, short-chain nucleic acids having an action of inhibiting expression of a target gene using an RNAi effect, and locked nucleic acids obtained by modifying aptamers and oligonucleotides.

### —Polypeptides—

Polypeptides mean polymers consisting of a plurality of amino acids. Of these, polypeptides which have a higher-order structure and exhibit a function derived from the higher-order structure are particularly called proteins. Both polypeptides unmodified from their naturally existing state and modified polypeptides are included in polypeptides. Modification includes acetylation, acylation, ADP-ribosylation, amidation, covalently bonding flavin thereto, covalently bonding a heme portion thereto, covalently bonding nucleotide or nucleotide derivatives thereto, covalently bonding lipids or lipid derivatives thereto, covalently bonding phosphatidylinositol thereto, cross-linking, cyclization, formation of disulfide bonds, demethylation, formation of covalent cross-links, formation of cystine, formation of pyroglutamate, formylation, γ-carboxylation, glycosylation, GPI anchor formation, hydroxylation, iodination, methylation, myristoylation, oxidation, a protein decomposition treatment, phosphorylation, prenylation, racemization, selenoylation, sulfation, transfer-RNA mediated addition of amino acids to proteins such as arginylation, ubiquitination, and the like. In a case where it is aimed to inhibit or suppress a function of a target protein, examples of proteins include a target protein variant having a dominant negative property with respect to target proteins and antibodies bonding to target proteins. Antibodies may be polyclonal antibodies or monoclonal antibodies as long as these bind to target proteins, or may be antibodies, such as bispecific antibodies or trispecific antibodies, which have multispecificity. Antibodies may be derived from any animal species as long as these exhibit a physiological effect, but are preferably human antibodies, human chimeric antibodies, or humanized antibodies. “Antibodies” of the present invention are typically immunoglobulin molecules such as IgG, IgE, IgM, IgA, and IgD. However, fragments of the antibodies (for example, F(ab′)2-fragments, Fab′ fragments, Fab fragments, Fv fragments, rIgG fragments, and single-chain antibodies) which have an antigen-binding region, or antibody-modified products (such as target antibodies) are also included therein as long as these bind to a specific antigen. Examples of other aspects of proteins include enzymes. Examples of enzymes include hydrolases, phosphatases, dephosphorylases, transferases, oxidoreductases, lyases, isomerases, and synthases.

Specific examples of proteins include quercetin, testosterone, indomethacin, tranilast, and tacrolimus.

### —Carbohydrates—

Examples of carbohydrates include monosaccharides, disaccharides, oligosaccharides, and polysaccharides. In addition, complex carbohydrates in which these carbohydrates are covalently bonded to proteins, lipids, or the like, or glycosides in which aglycones such as alcohols, phenols, saponins, and dyes bind to reducing groups of sugars are also included in carbohydrates.

### —Lipids—

Examples of lipids include simple lipids, complex lipids, and derived lipids.

### —Low-Molecular-Weight Compounds—

In general, natural or artificial substances having a molecular weight of several hundreds to several thousands are included in low-molecular-weight compounds. In addition, as low-molecular-weight compounds, there is a substance corresponding to the above-described poorly water-soluble substance and a substance corresponding to the above-described water-soluble substance. A low-molecular-weight compound may be in any form such as a salt or a hydrate as long as it functions as a physiologically active substance.

The poorly water-soluble substances are not particularly limited, and can be appropriately selected depending on the purpose. Examples thereof include griseofulvin, itraconazole, norfloxacin, tamoxifen, cyclosporine, glibenclamide, troglitazone, nifedipine, phenacetin, phenytoin, digitoxin, nilvadipine, diazepam, chloramphenicol, indomethacin, nimodipine, dihydroergotoxin, cortisone, dexamethasone, naproxen, turbuterol, beclomethasone propionate, fluticasone propionate, pranlukast, tranilast, loratidine, tacrolimus, amprenavir, bexarotene, calcitriol, clofazimine, digoxin, doxercalciferol, dronabinol, etoposide, isotretinoin, lopinavir, ritonavir, progesterone, saquinavir, sirolimus, tretinoin, amphotericin, fenoldopam, melphalan, paricalcitol, propofol, voriconazole, ziprasidone, docetaxel, haloperidol, lorazepam, teniposide, testosterone, and valrubicin. These may be used alone or in combination of two or more thereof.

The water-soluble substances are not particularly limited, and can be appropriately selected depending on the purpose. Examples thereof include abacavir, acetaminophen, acyclovir, amiloride, amitriptyline, antipyrine, atropine, buspirone, caffeine, captopril, chloroquine, chlorpheniramine, cyclophosphamide, diclofenac, desipramine, diazepam, diltiazem, diphenhydramine, disopyramide, doxin, doxycycline, enalapril, ephedrine, ethambutol, ethinyl estradiol, fluoxetine, imipramine, glucose, ketorol, ketoprofen, labetalol, levodopa, levofloxacin, metoprolol, metronidazole, midazolam, minocycline, misoprostol, metformin, nifedipine, phenobarbital, prednisolone, promazine, propranolol, quinidine, rosiglitazone, salicylic acid, theophylline, valproic acid, verapamil, and zidovudine. These may be used alone or in combination of two or more thereof.

Specific examples of low-molecular-weight compounds include kinase inhibitors such as gefitinib, erlotinib, osimertinib, bosutinib, vandetanib, alectinib, lorlatinib, abemaciclib, tyrphostin AG494, sorafenib, dasatinib, lapatinib, imatinib, motesanib, lestaurtinib, tandutinib, dorsomorphin, axitinib, and 4-benzyl-2-methyl-1,2,4-thiadiazolidine-3,5-dione.

### —Physiologically Active Substances Contained in Functional Food—

The physiologically active substances contained in functional foods are not particularly limited, and can be appropriately selected depending on the purpose. Examples thereof include vitamin A, vitamin D, vitamin E, lutein, zeaxanthin, lipoic acid, flavonoids, and fatty acids. These may be used alone or in combination of two or more thereof.

Examples of fatty acids include omega-3 fatty acids and omega-6 fatty acids.

### —Physiologically Active Substances Contained in Functional Cosmetics—

The physiologically active substances contained in functional cosmetics are not particularly limited, and can be appropriately selected depending on the purpose. Examples thereof include alcohols, fatty alcohols, polyols, aldehydes, alkanolamines, alkoxylated alcohols (for example, polyethylene glycol derivatives of alcohols, fatty alcohols, and the like), alkoxylated amides, alkoxylated amines, alkoxylated carboxylic acids, amides containing salts (for example, ceramides), amines, amino acids containing salts and alkyl-substituted derivatives, esters, alkyl-substituted and acyl derivatives, polyacrylic acids, acrylamide copolymers, adipic acid copolymers, amino silicones, biological polymers and derivatives thereof, butylene copolymers, carbohydrates (for example, polysaccharides, chitosan, and derivatives thereof), carboxylic acids, carbomers, esters, ethers, polymer ethers (for example, PEG derivatives and PPG derivatives), glyceryl esters and derivatives thereof, halogen compounds, heterocyclic compounds containing salts, hydrophilic colloids and derivatives thereof containing salts and rubber (for example, cellulose derivatives, gelatin, xanthan gum, and natural rubbers), imidazolines, inorganic substances (such as clay, TiO2, and ZnO), ketones (for example, camphor), isethionates, lanolin, and derivatives thereof, organic salts, phenols containing salts (for example, parabens), phosphorus compounds (for example, phosphoric acid derivatives), polyacrylates, acrylate copolymers, proteins, enzyme derivatives (for example, collagen), synthetic polymers containing salts, siloxanes, silanes, sorbitan derivatives, sterols, sulfonic acids and derivative thereof, and waxes. These may be used alone or in combination of two or more thereof.

A physiologically active substance preferably has a property such that the physiological activity is changed by heating, cooling, or external stress as described above. In a case of incorporating the physiologically active substance in the particles of the present invention, decrease in the amount of physiological activity of the produced particles is suppressed. Accordingly, based on the viewpoint that decrease in the amount of physiological activity can be suppressed, the effect of the present invention is exhibited significantly using a physiologically active substance, of which the physiological activity is easily changed by heating, cooling, or external stress, as a physiologically active substance contained in the particles of the present invention. Specifically, a physiologically active substance contained in a pharmaceutical composition is preferable as a physiologically active substance, at least one selected from proteins and nucleic acids is more preferable, and at least one selected from antibodies and enzymes is still more preferable.

The content of the physiologically active substance with respect to the total amount of particles is preferably 1 mass % to 95 mass % and more preferably 25 mass % to 75 mass %. In the case where the content thereof is greater than or equal to 25 mass %, sustained-release particles that stably release a physiologically active substance for a long period of time can be produced. The content of the physiologically active substance to be contained in the particles can be controlled by adjusting a formulation of a material mixture used when producing the particles. Particles having a higher content rate of a physiologically active substance than that of particles produced through other production methods can be produced through the production method of the present invention. In particular, it is possible to produce particles having a high content rate of a physiologically active substance through the production method in which granulation of particles is performed in a gas.

### —Others—

The particles of the present invention can contain any other components as materials constituting the particles as long as the components do not adversely affect final products such as pharmaceutical preparations, functional foods, and functional cosmetics. However, in consideration of biotoxicity, necessity of a removal step, or the like, it is preferable that the composition substantially not contain other components, such as, for example, a surfactant. For example, if substantially no surfactant is contained, it is possible to improve safety in a case where particles are contained in pharmaceutical compositions, functional foods, functional cosmetics, and the like. Examples of the case where other components are not substantially contained in particles include a case where the content of surfactant in the particles is less than or equal to a detection limit. Particles which do not substantially contain other components such as a surfactant can be realized, for example, by not using a surfactant during production of the particles (for example, by not blending a surfactant with a particle composition liquid). Particles which do not substantially contain a surfactant can be produced by removing a surfactant which has been used during the production of particles. However, since removing a surfactant sometimes change the physiological activity of a physiologically active substance or decreases the physiological activity ratio due to eluting of a physiologically active substance from particles, it is preferable not to use a surfactant during the production of particles.

### <Physical Properties of Particles>

Examples of characteristic physical properties of the particles of the present invention include a physiological activity ratio, a particle size distribution, and a particle diameter.

### —Physiological Activity Ratio—

A “physiological activity ratio” in the present application refers to a ratio ({amount of physiological activity after production of particles/amount of physiological activity before production of particles}×100) of the amount of physiological activity of particles made of materials used in production of the particles to the amount of physiological activity of the materials. In addition, the “amount of physiological activity” refers to a measurement value obtained when quantitatively measuring the physiological activity of a physiologically active substance. Here, “quantitative measurement” is not limited to a direct method in which the amount of physiological activity itself is quantitatively measured, and may be, for example, a relative, quantitative measurement method in which the amount of physiological activity is measured in comparison with a predetermined standard.

The particles of the present invention preferably have a property of a high physiological activity ratio. Specifically, the physiological activity ratio is preferably greater than or equal to 40%, more preferably greater than or equal to 50%, still more preferably greater than or equal to 60%, and particularly preferably greater than or equal to 70%.

Examples of factors that affect the physiological activity ratio include a physiological activity maintenance ratio and a physiologically active substance retention ratio. The physiological activity maintenance ratio refers to a proportion of physiological activity of a physiologically active substance maintained during producing particles. The physiological activity of a physiologically active substance is sometimes changed by heating, cooling, or external stress. Therefore, in a case where a treatment involving heating, cooling, shaking, stirring, or the like is executed in producing particles, the physiological activity maintenance ratio decreases. As a result, the physiological activity ratio also decreases. In addition, the physiologically active substance retention ratio refers to a proportion of a physiologically active substance held in particles during producing particles. For example, in some cases, a physiologically active substance may be lost due to decomposition or outflow in producing particles, and as a result, the total amount of physiologically active substance held in the particles may decrease. In such a case, the physiologically active substance retention ratio decreases, and as a result, the physiological activity ratio also decreases.

As methods for producing particles having a property of a high physiological activity ratio, a method for producing particles which does not involve decrease in activity due to heating, cooling, external stress, and/or the like is executed or a method for producing particles in which the amount of a physiologically active substance itself lost is low is executed, for example.

### —Particle Size Distribution—

The particles of the present invention preferably have a property of a narrow particle size distribution. Specific examples of factors representing the narrowness of the particle size distribution include a relative span factor (R.S.F) or volume-average particle diameter (Dv)/number-average particle diameter (Dn), and it is preferable that R.S.F be 0<(R.S.F)≤1.2 and volume-average particle diameter (Dv)/number-average particle diameter (Dn) be 1.00 to 1.50. By setting the particle size distribution within the above-described ranges, the proportion of particles corresponding to coarse particles decreases in terms of a target particle diameter. Accordingly, in a case where particles are contained in a pharmaceutical composition or the like, even if the pharmaceutical composition needs to be used after being subjected to filtration sterilization, it is possible to conveniently and efficiently perform the filtration sterilization without clogging a filtration sterilization filter. In addition, the uniform size of particles makes the contents of physiologically active substance and base material in each particle or the surface area of each particle uniform. Accordingly, the amount of physiologically active substance eluted from each of particles becomes equal, and therefore, it is possible to provide particles in which sustained releasability of a physiologically active substance is able to be controlled to a high degree. In addition, by making the size of particles uniform, it is possible to suppress generation of small diameter particles consisting of a physiologically active substance alone which is not contained in a base material, and to provide sustained-release particles in which an initial burst is inhibited.

### —Relative Span Factor (R.S.F)—

A “relative span factor (R.S.F)” in the present application is defined by (D90−D10)/D50. D90 represents a cumulative 90 volume % from a small particle side of a cumulative particle size distribution, D50 represents a cumulative 50 volume % from a small particle side of a cumulative particle size distribution, and D10 represents a cumulative 10 volume % from a small particle side of a cumulative particle size distribution. The (R.S.F) preferably satisfies 0<(R.S.F)≤1.2, more preferably satisfies 0<(R.S.F)≤1.0, and still more preferably satisfies 0<(R.S.F)≤0.6.

Examples of methods for measuring the (R.S.F) include a measurement method using Concentrated Particle Size Analyzer (“FPAR-1000”, manufactured by OTSUKA ELECTRONICS Co., LTD.) by a dynamic light scattering method.

### —Volume-average Particle Diameter (Dv)/Number-average Particle Diameter (Dn)—

Volume-average particle diameter (Dv)/number-average particle diameter (Dn) is a value obtained by dividing a volume-average particle diameter (Dv) by a number-average particle diameter (Dn). Volume-average particle diameter (Dv)/number-average particle diameter (Dn) is preferably 1.00 to 1.50 and more preferably 1.00 to 1.20.

Examples of methods for measuring a volume-average particle diameter (Dv) and a number-average particle diameter (Dn) include a measurement method using a laser diffraction-scattering-type particle size distribution measuring device (device name: Microtrack MT3000II, manufactured by MicrotracBEL Corp.)

### —Particle Diameter—

The volume-average particle diameter (Dv) of particles is preferably 10 nm to 100 μm, but can be appropriately selected depending on the purpose. A case where the volume-average particle diameter is 10 nm to 200 nm, a case where the volume-average particle diameter is 1 μm to 100 μm, and the like are more preferable.

In a case where the volume-average particle diameter (Dv) is 10 nm to 200 nm, when performing filtration sterilization on particles using a filtration sterilization filter, it is possible to suppress clogging of the filter. This is because a filter having a pore diameter of 220 nm is used for filtration sterilization performed on pharmaceutical compositions or the like. In addition, in a case where particles having a volume-average particle diameter (Dv) of 10 nm to 200 nm are used in a drug delivery system, it is also possible to selectively accumulate the particles in cancer tissues or the like using the particles having a specific particle diameter. The volume-average particle diameter (Dv) is more preferably 10 nm to 150 nm, still more preferably 10 nm to 100 nm, and particularly preferably 10 nm to 50 nm.

In a case where the volume-average particle diameter (Dv) is 1 μm to 100μ, it is possible to hold a sufficient amount of physiologically active substance. For example, it is possible to produce particles capable of performing sustained release of a physiologically active substance over a longer period of time. The volume-average particle diameter (Dv) is more preferably 1 μm to 50 μm, still more preferably 1 μm to 25 μm, and particularly preferably 1 μm 10 μm.

Examples of methods for measuring a volume-average particle diameter (Dv) of particles include a measurement method using Concentrated Particle Size Analyzer (“FPAR-1000”, manufactured by OTSUKA ELECTRONICS Co., LTD.) by a dynamic light scattering method, and a measurement method using a laser diffraction-scattering-type particle size distribution measuring device (device name: Microtrack MT3000H, manufactured by MicrotracBEL Corp.)

### <Use of Particles>

The particles of the present invention can be used, for example, in pharmaceutical compositions, functional foods, and functional cosmetics by combining with other components such as a dispersant and an additive as necessary. In addition, particles may be functional particles according to various applications. The functional fine particles are not particularly limited, and can be appropriately selected depending on the purpose. Examples thereof include immediate release particles, sustained-release particles, pH-dependent release particles, pH-independent release particles, enteric coated particles, controlled-release coated particles, and nanocrystal-containing particles.

### —Pharmaceutical Composition—

A pharmaceutical composition contains the particles of the present invention, and contains additive substances for preparations or the like as necessary. The additive substances are not particularly limited, and can be appropriately selected depending on the purpose. Examples of additive substances include excipients, a flavoring agent, a disintegrating agent, a fluidizer, an adsorbent, a lubricant, a corrigent, a surfactant, a flavoring, a colorant, an antioxidant, a masking agent, antistatic agent, and a moistening agent. These may be used alone or in combination of two or more thereof.

### —Excipients—

The excipients are not particularly limited, and can be appropriately selected depending on the purpose. Examples thereof include lactose, sucrose, mannitol, glucose, fructose, maltose, erythritol, maltitol, xylitol, palatinose, trehalose, sorbitol, crystalline cellulose, talc, anhydrous silicic acid, anhydrous calcium phosphate, precipitated calcium carbonate, and calcium silicate. These may be used alone or in combination of two or more thereof.

### —Flavoring Agents—

The flavoring agents are not particularly limited, and can be appropriately selected depending on the purpose. Examples thereof include L-menthol, white sugar, D-sorbitol, xylitol, citric acid, ascorbic acid, tartaric acid, malic acid, aspartame, acesulfame potassium, thaumatin, saccharin sodium, dipotassium glycyrrhizin, sodium glutamic acid, 5′-sodium inosinate, and 5′-sodium guanylate. These may be used alone or in combination of two or more thereof.

### —Disintegrators—

The disintegrators are not particularly limited, and can be appropriately selected depending on the purpose. Examples thereof include low-substituted hydroxypropyl cellulose, carmellose, carmellose calcium, sodium carboxymethyl starch, sodium croscarmellose, crospovidone, hydroxypropyl starch, and corn starch. These may be used alone or in combination of two or more thereof.

### —Fluidizers—

The fluidizers are not particularly limited, and can be appropriately selected depending on the purpose. Examples thereof include light anhydrous silicic acid, hydrated silicon dioxide, and talc. These may be used alone or in combination of two or more thereof.

Commercially available products can be used as light anhydrous silicic acid. Commercially available products of light anhydrous silicic acid are not particularly limited, and can be appropriately selected depending on the purpose. Examples thereof include Adsolider 101 (manufactured by Freund Corporation; average pore diameter of 21 nm).

### —Adsorbents—

Commercially available products can be used as adsorbents. Commercially available products of adsorbents are not particularly limited, and can be appropriately selected depending on the purpose. Examples thereof include a trade name of Carprex (component name of synthetic silica, registered trademark of DSL. Japan Co., Ltd.), a trade name of Aerosil (registered trademark of NIPPON AEROSIL CO., LTD.) 200 (component name of hydrophilic fumed silica), a trade name of Silysia (component name of amorphous silicon dioxide, registered trademark of Fuji Silysia Chemical Ltd.), and a trade name of Alcamac (component name of synthetic hydrotalcite, registered trademark of Kyowa Chemical Industry Co., Ltd.) These may be used alone or in combination of two or more thereof.

### —Lubricants—

The lubricants are not particularly limited, and can be appropriately selected depending on the purpose. Examples thereof include magnesium stearate, calcium phosphate, sucrose fatty acid esters, sodium stearyl fumarate, stearic acid, polyethylene glycol, and talc. These may be used alone or in combination of two or more thereof.

### —Corrigents—

The corrigents are not particularly limited, and can be appropriately selected depending on the purpose. Examples thereof include trehalose, malic acid, maltose, potassium gluconate, anise essential oil, vanilla essential oil, and cardamom essential oil. These may be used alone or in combination of two or more thereof.

### —Surfactants—

The surfactants are not particularly limited, and can be appropriately selected depending on the purpose. Examples thereof include polysorbates such as polysorbate 80; a polyoxyethylene-polyoxypropylene copolymer; and sodium lauryl sulfate. These may be used alone or in combination of two or more thereof.

### —Flavorings—

Flavorings are not particularly limited, and can be appropriately selected depending on the purpose. Examples thereof include lemon oil, orange oil, and peppermint oil. These may be used alone or in combination of two or more thereof.

### —Colorants—

The colorants are not particularly limited, and can be appropriately selected depending on the purpose. Examples thereof include titanium oxide, Food Yellow No. 5, Food Blue No. 2, iron sesquioxide, and yellow iron sesquioxide. These may be used alone or in combination of two or more thereof.

### —Antioxidants—

The antioxidants are not particularly limited, and can be appropriately selected depending on the purpose. Examples thereof include sodium ascorbate, L-cysteine, sodium sulfite, and vitamin E. These may be used alone or in combination of two or more thereof.

### —Masking Agents—

The masking agents are not particularly limited, and can be appropriately selected depending on the purpose. Examples thereof include titanium oxide. These may be used alone or in combination of two or more thereof.

### —Antistatic Agents—

The antistatic agents are not particularly limited, and can be appropriately selected depending on the purpose. Examples thereof include talc and titanium oxide. These may be used alone or in combination of two or more thereof.

### —Moistening Agents—

The moistening agents are not particularly limited, and can be appropriately selected depending on the purpose. Examples thereof include polysorbate 80, sodium lauryl sulfate, sucrose fatty acid esters, macrogol, and hydroxypropyl cellulose (HPC). These may be used alone or in combination of two or more thereof.

Preparations of pharmaceutical compositions are not particularly limited, and can be appropriately selected depending on the purpose. Examples thereof include a large intestine delivery preparation, a lipid microsphere preparation, a dry emulsion preparation, a self-emulsifying preparation, dry syrup, a powder preparation for nasal administration, a powder preparation for pulmonary administration, a wax matrix preparation, a hydrogel preparation, a polymeric micelle preparation, a mucoadhesive preparation, a gastric floating preparation, a liposome preparation, and a solid dispersion preparation. These may be used alone or in combination of two or more thereof.

Examples of dosage forms of pharmaceutical compositions include tablets, capsules, suppositories, and other solid dosage forms; aerosols for intranasal or pulmonary administration; and liquid agents such as an injection agent, an intraocular agent, an intra-air agent, and oral agent.

Administration route of pharmaceutical compositions are not particularly limited, and can be appropriately selected depending on the purpose. Examples thereof include oral administration, intranasal administration, rectal administration, vaginal administration, subcutaneous administration, intravenous administration, and pulmonary administration. Among these, oral administration is preferable.

### —Functional Foods—

A functional food contains the particles of the present invention and a food, and contains other additive substances as necessary.

Foods are not particularly limited, and can be appropriately selected depending on the purpose. Examples thereof include frozen desserts, noodles, confectioneries, seafood, seafood and livestock processed foods, dairy products, oils and fats, oil and fat processed foods, seasonings, retort pouch foods, health foods, and nutritional supplements.

### —Functional Cosmetics—

Functional cosmetics contain the particles of the present invention and cosmetics, and contain other-additive substances as necessary.

Cosmetics are not particularly limited, and can be appropriately selected depending on the purpose. Examples thereof include skin care cosmetics, makeup cosmetics, hair care cosmetics, body care cosmetics, and fragrance cosmetics.

### <<Device and Method for Producing Particles>>

The method for producing particles of the present invention includes: discharging liquid droplets containing a base material, a physiologically active substance having physiological activity, and a solvent; and granulating particles by removing the solvent from the liquid droplets, and includes other steps as necessary.

The particle production device of the present invention includes liquid droplet discharge means for discharging liquid droplets containing a base material, a physiologically active substance having physiological activity, and a solvent; and granulation means for granulating particles by removing the solvent from the liquid droplets, and includes other means as necessary.

“Removal” in the present application means that a solvent contained in a liquid phase is removed from the liquid phase, but is not limited to a case where all the solvent contained in the liquid phase is removed. The solvent contained in the liquid phase may remain as long as particles can be granulated. In addition, “removal” in the present application is not particularly limited as long as a solvent contained in a liquid phase is removed from the liquid phase. For example, there is an aspect in which a liquid phase is brought into contact with another liquid phase to diffuse a solvent contained in the liquid phase in the other liquid phase (hereinafter also referred to as “in-liquid drying”) and an aspect in which a solvent contained in a liquid phase is vaporized from the liquid phase in a gas or in vacuum (hereinafter, also referred to as “in-gas drying”.)

Next, an example of a case where removal is performed through in-liquid drying will be described as a first embodiment, and an example of a case where removal is performed through in-gas drying will be described as a second embodiment. However, the method and device for producing particles are not limited to these embodiments.

### <Method for Producing Particles as First Embodiment>

A method for producing particles as a first embodiment (in-liquid drying) includes: discharging liquid droplets containing a base material, a physiologically active substance having physiological activity, and a good solvent of the base material into a poor solvent of the base material; and granulating particles by bringing the liquid droplets into contact with the poor solvent and removing the good solvent which has been contained in the liquid droplets, and includes other steps as necessary.

A plurality of methods are known in the related art as a wet-type granulation method, such as the first embodiment, in which particles are granulated in a liquid.

Examples thereof include a in-liquid pulverization method such as a method for obtaining pulverized particles, of which the particle diameter is reduced, by stirring a particle material in a liquid under a high shear force using a media-type stirrer or a medialess-type stirrer.

In addition, more examples thereof include a two-liquid mixing method such as a method for obtaining precipitated particles, of which the particle diameter is reduced, by adding a good solvent containing a dissolved particle material to a poor solvent which has been stirred under a high shear force with a mixing stirrer, or a method for obtaining dispersed particles, of which the particle diameter is reduced, by adding a liquid containing a particle material and a solvent in which the particle material is dissolved to an aqueous medium which has been stirred under a high shear force with a mixing stirrer in the presence of a surfactant.

The in-liquid pulverization method can produce particles with a small particle diameter, but it is difficult to produce particles with a narrow particle size distribution. In addition, in a case where a media-type stirrer is used, particles contain impurities derived from media, and in some cases, it may be difficult to contain particles in pharmaceutical compositions, functional foods, functional cosmetics, and the like. In addition, in a case where a medialess-type stirrer is used, productivity may deteriorate. Furthermore, in the in-liquid pulverization method, since a large external stress is generated by stirring with a high shear force in a pulverization process, in a case where a physiologically active substance having a property such that physiological activity is changed by the external stress is contained as a material of particles, the physiological activity of the physiologically active substance changes, and as a result, the amount of physiological activity decreases.

The two-liquid mixing method can produce particles with a narrow particle size distribution, but a surfactant sometimes remain in the produced particles. In some cases, it is difficult to incorporate the particles in pharmaceutical compositions, functional foods, functional cosmetics, and the like depending on the type of surfactant. In addition, it is also possible to perform a treatment of removing the surfactant from the particles. However, in a case where a physiologically active substance has a property such that physiological activity is changed by heating, cooling, or external stress, the physiological activity is changed by the treatment, and as a result, the amount of physiological activity sometimes decreases. Furthermore, in the two-liquid mixing method, since a large external stress is generated by stirring with a high shear force in a mixing and stirring process, in a case where a physiologically active substance having a property such that physiological activity is changed by the external stress is contained as a material of particles, the physiological activity of the physiologically active substance changes, and as a result, the amount of physiological activity decreases.

Since the method for producing particles as the first embodiment to be described below does not correspond to the above-described in-liquid pulverization method and the two-liquid mixing method, even in the case where a physiologically active substance having a property such that physiological activity is changed by heating, cooling, or external stress is contained as a material of particles, the change in physiological activity of the physiologically active substance is suppressed, and as a result, the decrease in amount of physiological activity is also suppressed.

In addition, the method for producing particles as the first embodiment preferably does not contain using means such as shaking or stirring for imparting external stress to such a degree that physiological activity of a physiologically active substance changes, using heating means for controlling the temperature so that physiological activity of a physiologically active substance changes, and using cooling means for controlling the temperature so that physiological activity of a physiologically active substance changes.

### —Liquid Droplet Discharge Step—

The liquid droplet discharge step of the first embodiment is discharging liquid droplets containing a base material, a physiologically active substance having physiological activity, and a good solvent of the base material into a poor solvent of the base material.

The method for discharging liquid droplets is not particularly limited, but examples thereof include the following methods.

(i) Method using discharge means for discharging liquid under pressure as liquid droplets from holes provided on flat plate-shaped nozzle formation surface of ink jet nozzle or the like

(ii) Method using discharge means for discharging liquid under pressure as liquid droplets from holes with irregular shape of SPG film

(iii) Method using Discharge means for discharging liquid, to which vibration is applied, as liquid droplets from holes

Examples of the above-described discharge means of (iii) in which vibration is used and physiological activity of a physiologically active substance is not changed by the vibration include means which hardly applies external stress to a particle composition liquid itself, for example, discharge means using a film vibration method, discharge means using a Rayleigh division method, discharge means using a liquid vibration method, and discharge means using a liquid column resonance method. In addition, such means may further have means for discharging a liquid by applying pressure to the liquid. Among these means, discharge means in which a liquid column resonance method is used and which further has means for discharging a liquid by applying pressure to the liquid is preferable.

Examples of discharge means using a liquid column resonance method include discharge means in which a method for applying vibration to a liquid accommodated in a liquid column resonance liquid chamber to form a standing wave due to liquid column resonance, and discharging the liquid from discharge holes formed in an amplitude direction of the standing wave into a region corresponding to an antinode of the standing wave is used.

Examples of discharge means using a film vibration method include discharge means disclosed in Japanese Unexamined Patent Application, First Publication No. 2008-292976. Examples of discharge means using a Rayleigh division method include discharge means disclosed in Japanese Patent No. 4647506. Examples of discharge means using a liquid vibration method include discharge means disclosed in Japanese Unexamined Patent Application, First Publication No. 2010-102195.

The diameter of a discharge hole which is provided in the above-described discharge means and discharges liquid droplets is preferably less than 1,000 μm, more preferably greater than or equal to 1.0 μm and less than 1,000 μm, still more preferably 1.0 μm to 500 μm, and particularly preferably 1.0 μm to 50 μm. In a case where the shape of a discharge hole is not a perfect circle, a diameter of a perfect circle having the same area as that of the discharge hole is employed.

In the liquid droplet discharge step, discharge may be performed in a state in which discharge holes are in a poor solvent (in other words, a state in which the discharge holes come into contact with the poor solvent), or in a state in which discharge holes are not in a poor solvent (in other words, a state in which the discharge holes do not come into contact with the poor solvent). However, the discharge is preferably performed in a state in which discharge holes are in a poor solvent. By performing discharge in a state in which discharge holes are in a poor solvent, it is possible to suppress drying of a discharged liquid in the discharge holes and to suppress discharge failure. In addition, it is possible to make a particle size distribution of particles to be produced narrower.

In the case where discharge holes are in a poor solvent, the length from the liquid level of the poor solvent to the discharge holes (in other words, the immersive length of the discharge holes in the poor solvent) is not particularly limited, and can be appropriately selected depending on the purpose. 1.0 mm to 10 mm is preferable and 2.0 mm to 5.0 mm is more preferable.

A liquid (particle composition liquid) discharged in the liquid droplet discharge step contains a base material, a physiologically active substance having physiological activity, and a good solvent of the base material and is discharged into a poor solvent. Since various materials similar to those of the base material and the physiologically active substance contained in particles can be used as the base material and the physiologically active substance contained in the liquid, description thereof will not be repeated, and only the good solvent and the poor solvent will be described.

The particle composition liquid and the poor solvent do not substantially contain a surfactant. If substantially no surfactant is contained, it is possible to improve safety in a case where particles to be produced are contained in pharmaceutical compositions, functional foods, functional cosmetics, and the like. Here, examples of the case where a surfactant is not substantially contained include a case where the content of surfactant in a particle composition liquid and a poor solvent is less than or equal to a detection limit and a case where no surfactant is contained in a particle composition liquid and a poor solvent.

### —Good Solvent—

A “good solvent” in the present application means a solvent in which the solubility of a base material is high. In addition, a good solvent can be defined by the mass of base material that can be dissolved in 100 g of a solvent at a temperature of 25° C. For example, the mass of a base material that can be dissolved is preferably greater than or equal to 0.10 g.

A good solvent is preferably a solvent in which the solubility of a physiologically active substance is high. Similarly to the base material, the mass of a physiologically active substance that can be dissolved is preferably greater than or equal to 0.10 g, for example.

The good solvent is not particularly limited, and can be appropriately selected depending on the purpose. Examples thereof include alcohols, ketones, ethers, acetonitrile, and tetrahydrofuran. Examples of alcohols include alcohols having 1 to 4 carbon atoms. Examples of alcohols having 1 to 4 carbon atoms include methanol, ethanol, propanol, and butanol. Examples of ketones include ketones having 3 to 6 carbon atoms. Examples of ketones having 3 to 6 carbon atoms include acetone, methyl ethyl ketone, and cyclohexanone. Examples of ethers include ethers having 2 to 6 carbon atoms. Examples of ethers having 2 to 6 carbon atoms include dimethyl ether, methyl ethyl ether, and diethyl ether. These may be used alone or in combination of two or more thereof. Among these, a good solvent in which alcohols and ketones are used in combination is preferable, and a good solvent in which ethanol and acetone are used in combination is more preferable.

The content of base material in a good solvent is not particularly limited, and can be appropriately selected depending on the purpose. In a case where a good solvent in which acetones and ethanol are used in combination is used, the content of base material based on the mass of a particle composition liquid is preferably less than or equal to 5.0 mass % and more preferably 0.1 mass % to 5.0 mass %. In the case where the content of base material is less than or equal to 5.0 mass %, a particle size distribution of particles becomes narrow. The particle diameter of particles to be produced can be controlled by adjusting the content of base material.

### —Poor Solvent—

A “poor solvent” in the present application means a solvent in which the solubility of a base material is low or a solvent in which a base material does not dissolve. A poor solvent can be defined by the mass of base material that can be dissolved in 100 g of a solvent at a temperature of 25° C. For example, the mass of a base material that can be dissolved is preferably less than or equal to 0.05 g.

A poor solvent is preferably a solvent in which the solubility of a physiologically active substance is low. Similarly to the base material, the mass of a physiologically active substance that can be dissolved is preferably less than or equal to 0.05 g, for example.

The poor solvents are not particularly limited, and can be appropriately selected depending on the purpose. For example, water is preferable.

A stabilizer may be contained in a poor solvent for the purpose of suppressing aggregation of produced particles and crystal growth of a physiologically active substance. The stabilizers are not particularly limited, and can be appropriately selected depending on the purpose. Examples thereof include polyethylene glycol fatty acid esters, sorbitan fatty acid esters, polyoxyethylene hydrogenated castor oil, polyoxyethylene alkyl ether, quaternary ammonium salts, lecithin, polyvinyl pyrrolidone, polyvinyl alcohols, glycerides, fatty acids, and steroids. Among these, polyethylene glycol fatty acid esters, sorbitan fatty acid esters, polyvinyl pyrrolidone, polyvinyl alcohols, glycerides, fatty acids, steroids, and phospholipids are preferable. Furthermore, polyethylene glycol fatty acid esters, sorbitan fatty acid esters, and fatty acids are more preferable. Specifically, polyoxyl 40 stearate, polysorbate 80, and stearic acid are preferable. These may be used alone or in combination of two or more thereof.

In addition, the content of stabilizer to be added is preferably less than or equal to 5 mass % based on the mass of a poor solvent.

By incorporating a poor solvent into a stabilizer, the surface of particles formed in the poor solvent is coated with a stabilizer to form a hydrophilic coating layer, and the particles are easily incorporated into a living body. The coating may be complete coating or partial coating.

### —Granulation Step—

The granulation step of the first embodiment is granulating particles by bringing liquid droplets into contact with a poor solvent and removing a good solvent which has been contained in the liquid droplets. Specifically, the granulation step is granulating particles by bringing liquid droplets discharged into a poor solvent through the above-described liquid droplet discharge step into contact with the poor solvent so that the good solvent contained in the liquid droplets and the poor solvent are mutually diffused to precipitate the particles due to supersaturated base material contained in the liquid droplets. By granulating particles through this step, it is possible to make the form of the particles into a solid dispersion and specifically to produce particles in a form in which a physiologically active substance is dispersed in a base material.

Unlike the method in the related art, the particles produced through this method are granulated by bringing the liquid droplets into contact with the poor solvent, and therefore, a particle size distribution can be made narrow. In addition, the particle diameter of particles can also be adjusted by appropriately adjusting the size or the like of discharge holes of discharge means that forms liquid droplets. In a case where liquid droplet formation with discharge means is used as means for reducing the particle diameter of particles instead of using a stirrer performing stirring or the like under a high shear force, even in the case where a physiologically active substance having a property such that physiological activity is changed by external stress is contained as a material of the particles, the change in physiological activity of the physiologically active substance is suppressed, and as a result, the decrease in amount of physiological activity is also suppressed. Accordingly, it is possible to increase the physiological activity ratio of the particles through this step compared to the method in the related art, for example, it is possible to make the physiological activity ratio greater than or equal to 50%.

In the granulation step, a poor solvent is preferably fluidized. The flow rate is not particularly limited as long as a strong external stress that affects physiological activity of a physiologically active substance is not generated, but is preferably greater than or equal to 0.3 m/s and more preferably 1.0 m/s, for example. By fluidizing a poor solvent, it is possible to suppress coalescence of particles and to make a particle size distribution narrow.

In the granulation step, a poor solvent is preferably circulating. The circulation is preferably performed by providing a circulation path in a poor solvent accommodation container that accommodates a poor solvent. By circulating the poor solvent, it is possible to suppress coalescence of particles and to make a particle size distribution narrow.

In the granulation step, it is preferable that a good solvent accumulated in a poor solvent due to discharged liquid droplets be removed. By removing the good solvent in the poor solvent, it is possible to suppress coalescence of particles and to make a particle size distribution narrow.

### —Other Steps—

Examples of other steps include a good solvent removal step, a filtration sterilization step, and a poor solvent removal step.

### —Good Solvent Removal Step—

The good solvent removal step is not particularly limited as long as it is removing a good solvent from a liquid containing produced particles, and can be appropriately selected depending on the purpose. Examples thereof include a method for obtaining a suspension containing particles by subjecting a liquid containing the particles to a decompression treatment to volatilize a good solvent.

### —Filtration Sterilization Step—

The filtration sterilization step is not particularly limited as long as it is filtering the suspension containing particles after the good solvent removal step using a sterilization filter, and can be appropriately selected depending on the purpose. In addition, the suspension which contains particles and is subjected to filtration may or may not be diluted with a poor solvent.

The sterilization filter is not particularly limited, and can be appropriately selected depending on the purpose. Examples thereof include a nylon membrane filter. The filtration accuracy of a sterilization filter is not particularly limited, and can be appropriately selected depending on the purpose, but is preferably 0.1 μm to 0.45 μm. A commercially available product may be used as a sterilization filter. Examples of commercially available products include a LifeASSURE™ nylon membrane filter cartridge (with a filtration accuracy of 0.1 μm).

### —Poor Solvent Removal Step—

The poor solvent removal step is not particularly limited as long as it is removing a poor solvent from a filtrate after filtration sterilization, and can be appropriately selected depending on the purpose. Examples thereof include a method for removing a poor solvent through filtration.

### <Particle Production Device as First Embodiment>

A particle production device as a first embodiment (in-liquid drying) includes: liquid droplet discharge means for discharging liquid droplets containing a base material, a physiologically active substance having physiological activity, and a good solvent of the base material into a poor solvent of the base material; and granulation means for granulating particles by bringing the liquid droplets into contact with the poor solvent and removing the good solvent which has been contained in the liquid droplets, and includes other means as necessary.

### —Liquid Accommodation Container—

A liquid accommodation container is a container in which a liquid containing a base material, a physiologically active substance, and a good solvent is accommodated. The liquid accommodation container may or may not be flexible. The material of the liquid accommodation container is not particularly limited, and can be appropriately selected depending on the purpose. For example, the material may be made of resin or metal. The structure of the liquid accommodation container is not particularly limited, and can be appropriately selected depending on the purpose. For example, the structure may be a closed structure or may be a non-closed structure.

### —Liquid Droplet Discharge Means—

The liquid droplet discharge means is means for discharging a liquid containing a base material, a physiologically active substance, and a good solvent to form liquid droplets.

The liquid droplet discharge means is connected to the liquid accommodation container. Means for connecting the liquid droplet discharge means to the liquid accommodation container is not particularly limited as long as a liquid can be supplied from the liquid accommodation container to the liquid droplet discharge means, and can be appropriately selected depending on the purpose. Examples thereof include a pipe (such as a tube).

The liquid droplet discharge means is not particularly limited as long as it is means that can discharge liquid droplets, but preferably has a vibration-applying member that discharges liquid droplets by applying vibration to a liquid. The vibration is not particularly limited, and can be appropriately selected depending on the purpose. For example, the frequency is preferably greater than or equal to 1 kHz, more preferably greater than or equal to 150 kHz, and still more preferably 300 kHz to 500 kHz. It is possible to reproducibly make a liquid column injected from discharge holes into liquid droplets in a case where the vibration is greater than or equal to 1 kHz, and it is possible to improve the production efficiency in a case where the vibration is greater than or equal to 150 kHz.

Examples of the liquid droplet discharge means having a vibration-applying member include an ink jet nozzle. A liquid column resonance method, a film vibration method, a liquid vibration method, and a Rayleigh division method can be used as discharge mechanism of an ink jet nozzle, for example.

### —Granulation Means—

The granulation means is means for granulating particles by bringing liquid droplets into contact with a poor solvent and removing a good solvent which has been contained in the liquid droplets.

Examples of granulation means include a poor solvent accommodation container in which a poor solvent is accommodated. The poor solvent accommodation container may or may not be flexible. The material of the poor solvent accommodation container is not particularly limited, and can be appropriately selected depending on the purpose. For example, the material may be made of resin or metal.

The granulation means preferably has fluidizing means for fluidizing a poor solvent.

The granulation means preferably has a circulation path provided with circulation means for circulating the poor solvent accommodated therein. The circulation path may be, for example, a circulation path configured to have a pipe or a circulation path having a pipe and a tank.

The granulation means preferably has good solvent removal means for removing a good solvent accumulated in a poor solvent due to discharged liquid droplets. Examples of good solvent removal means include decompression means.

### Specific Example of First Embodiment

Next, a specific aspect of the first embodiment will be described based on an aspect in which liquid column resonance liquid droplet discharge means is used as liquid droplet discharge means. As a matter of course, those skilled in the art understand that liquid droplet discharge means is not limited to liquid column resonance liquid droplet discharge means and other types of liquid droplet discharge means (for example, discharge means using a film vibration method, discharge means using a Rayleigh division method, and discharge means using a liquid vibration method) may be used.

First, liquid column resonance liquid droplet discharge means, which is one type of means constituting a particle production device, will be specifically described.

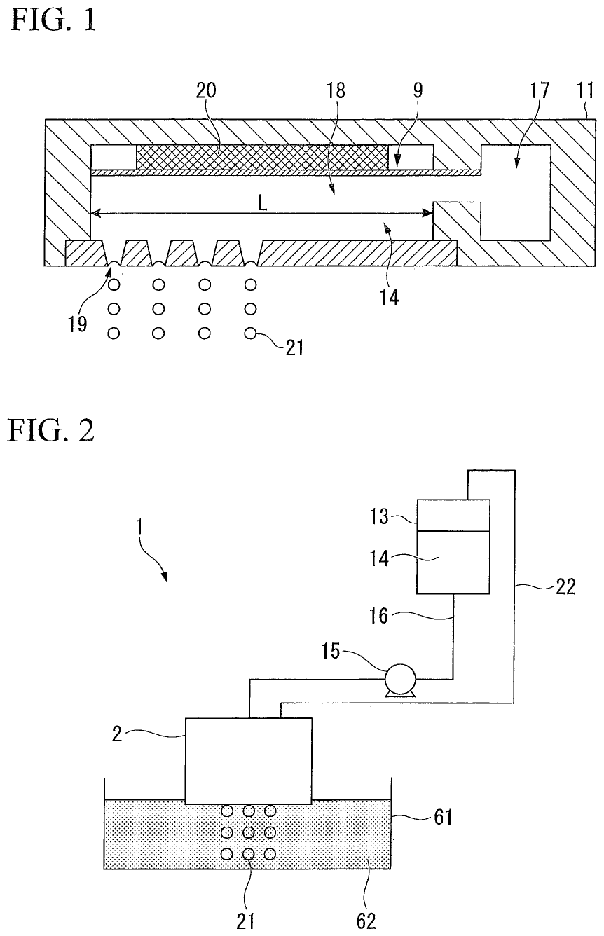

FIG. 1 is a schematic cross-sectional view showing an example of liquid column resonance liquid droplet discharge means. Liquid column resonance liquid droplet discharge means 11 includes a liquid common supply path 17 and a liquid column resonance liquid chamber 18. The liquid column resonance liquid chamber 18 communicates with the liquid common supply path 17 provided on one of wall surfaces at both ends in a longitudinal direction. In addition, the liquid column resonance liquid chamber 18 includes: discharge holes 19 that discharge liquid droplets 21 on one of wall surfaces connected to the wall surfaces at both ends; and vibration generation means 20 which is provided on a wall surface opposite to the discharge holes 19 and generates high-frequency vibration for forming liquid column resonance standing waves. A high-frequency power source is connected to the vibration generation means 20. In addition, an airflow passage for supplying airflow conveying the liquid droplets 21 discharged from liquid column resonance liquid droplet discharge means 11 may be provided.

A liquid 14 containing a base material, a physiologically active substance, and a good solvent flows into the liquid common supply path 17 of the liquid column resonance liquid droplet discharge means 11 through a liquid supply pipe due to a liquid circulation pump, and is supplied to the liquid column resonance liquid chamber 18. In the liquid column resonance liquid chamber 18 filled with the liquid 14, pressure distribution is formed by a liquid column resonance standing wave generated by the vibration generation means 20. The liquid droplets 21 are discharged from the discharge holes 19 arranged in a region which is a portion with a large amplitude in the liquid column resonance standing wave, has large pressure fluctuation, and corresponds to an antinode of the standing wave. The region corresponding to an antinode of the standing wave due to this liquid column resonance is a region other than a node of the standing wave. A region having a sufficiently large amplitude so that the pressure fluctuation of the standing wave discharges a liquid is preferable, and a region of ±¼ wavelength from the position (node as a velocity standing wave) at which the amplitude of the pressure stationary wave becomes maximum to the position at which the amplitude of the pressure stationary wave becomes minimum is more preferable.

In the case of the region corresponding to an antinode of a standing wave, even if a plurality of discharge holes are open, it is possible to form approximately uniform liquid droplets from the discharge holes and to efficiently discharge the liquid droplets. As a result, clogging of the discharge holes hardly occurs. The liquid 14 passing through the liquid common supply path 17 circulates through a liquid return pipe. In a case where the amount of liquid 14 in the liquid column resonance liquid chamber 18 decreases due to discharge of the liquid droplets 21, a suction force due to an action of the liquid column resonance standing wave in the liquid column resonance liquid chamber 18 acts, and the flow rate of the liquid 14 supplied from the liquid common supply path 17 increases. Then, the liquid column resonance liquid chamber 18 is supplemented with the liquid 14. In the case where the liquid column resonance liquid chamber 18 is supplemented with the liquid 14, the flow rate of the liquid 14 passing through the liquid common supply path 17 returns to its original level.

The liquid column resonance liquid chamber 18 of the liquid column resonance liquid droplet discharge means 11 is formed by joining frames which are made of materials, such as metal, ceramics, and silicone, with high rigidity to such a degree that the resonance frequency of the liquid at a driving frequency is not influenced. In addition, as shown in FIG. 1, a length L between the wall surfaces at both ends of the liquid column resonance liquid chamber 18 in the longitudinal direction is determined based on a liquid column resonance principle. Furthermore, it is preferable that a plurality of liquid column resonance liquid chambers 18 be arranged in one liquid droplet formation unit for dramatically improving productivity. The number of liquid column resonance liquid chambers 18 is not particularly limited, and 1 to 2,000 is preferable. In addition, a flow path for liquid supply is communicatively connected to each liquid column resonance liquid chamber from the liquid common supply path 17, and a plurality of liquid column resonance liquid chambers 18 communicate with the liquid common supply path 17.

In addition, the vibration generation means 20 of the liquid column resonance liquid droplet discharge means 11 is not particularly limited as long as it can be driven at a predetermined frequency, but a form in which a piezoelectric body is stuck on an elastic plate 9 is preferable. From the viewpoint of productivity, the frequency is more preferably greater than or equal to 150 kHz, and still more preferably 300 kHz to 500 kHz. The elastic plate constitutes a part of the wall of the liquid column resonance liquid chamber so that the piezoelectric body does not come into contact with a liquid. Examples of piezoelectric bodies include piezoelectric ceramics such as lead zirconate titanate (PZT). In many cases, piezoelectric bodies are stacked when in use since these generally have a small displacement amount. In addition, other examples thereof include piezoelectric polymers such as polyvinylidene fluoride (PVDF), and single crystals such as crystal, LiNbO3, LiTaO3, and KNbO3. Furthermore, the vibration generation means 20 is preferably disposed in each liquid column resonance liquid chamber so as to individually control the liquid column resonance liquid chamber. In addition, a configuration is preferable in which liquid column resonance liquid chambers can be individually controlled through an elastic plate by partially cutting a block-shaped vibration member made of one of the above-described materials along with the arrangement of the liquid column resonance liquid chambers.

Furthermore, from the viewpoints that a large number of openings of the discharge holes 19 can be provided to increase the production efficiency, it is preferable to employ a configuration in which the discharge holes 19 are provided in the liquid column resonance liquid chambers 18 in the width direction. In addition, since the liquid column resonance frequency varies depending on the arrangement of the openings of the discharge holes 19, it is desirable to appropriately determine the liquid column resonance frequency while checking discharge of liquid droplets.

Next, the particle production device will be specifically described.

FIG. 2 is a schematic diagram showing an example of a particle production device. A particle production device 1 includes a liquid accommodation container 13, liquid droplet discharge means 2, and a poor solvent accommodation container 61. The liquid accommodation container 13 accommodating the liquid 14 and a liquid circulation pump 15, which supplies the liquid 14 accommodated in the liquid accommodation container 13 to the liquid droplet discharge means 2 through a liquid supply pipe 16 and pressure-feeds the liquid 14 in the liquid supply pipe 16 to return the liquid to the liquid accommodation container 13 through a liquid return pipe 22, are connected to the liquid droplet discharge means 2, and the liquid 14 can be supplied to the liquid droplet discharge means 2 at all times.

The liquid droplet discharge means 2 includes, for example, the liquid column resonance liquid droplet discharge means 11 shown in FIG. 1.

The liquid 14 is discharged as the liquid droplets 21 from the liquid droplet discharge means 2 into a poor solvent 62 accommodated in the poor solvent accommodation container 61.

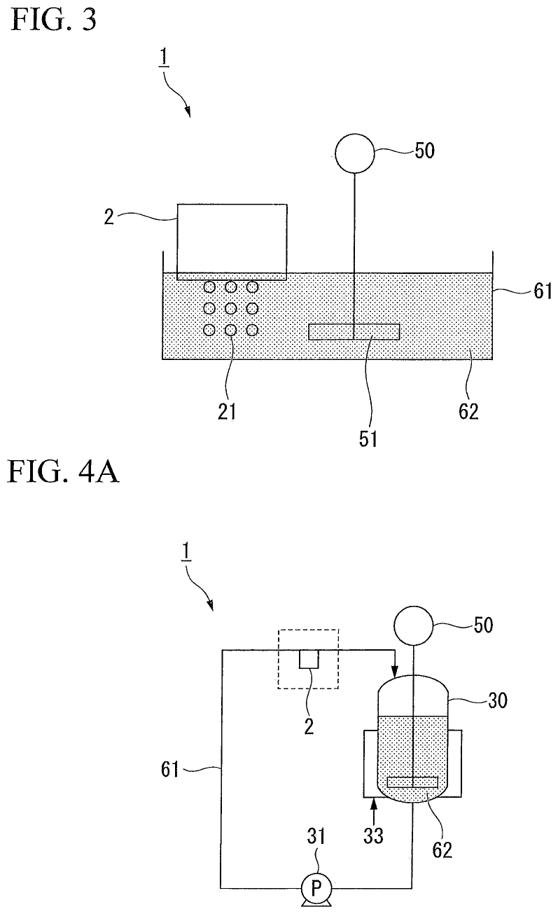

FIG. 3 is a schematic diagram showing another example of a particle production device.

A particle production device 1 of FIG. 3 is a schematic diagram in a case of discharging a liquid into a poor solvent 62 in a poor solvent accommodation container 61 which is a glass container. A discharge unit of liquid droplet discharge means 2 discharges a liquid into the poor solvent 62 in a state of being immersed in the poor solvent 62.

The particle production device 1 of FIG. 3 includes a stirring member 50 having a stirring blade 51. The stirring blade 51 is immersed in the poor solvent 62 in the poor solvent accommodation container 61.

When liquid droplets 21 are discharged into the poor solvent 62 through the liquid droplet discharge means 2, coalescence of particles formed by the liquid droplets 21 is prevented by rotating the stirring blade 51 to stir the poor solvent 62. The stirring with the stirring member 50 is performed within a range where physiological activity of a physiologically active substance contained in particles does not change. In addition, the stirring may not be performed.

Next, another example of the particle production device will be described.

As a method for preventing coalescence of particles formed by liquid droplets coming into contact with a poor solvent, it is preferable to impart fluidity, which is a flow of the poor solvent, to a discharge unit of liquid droplet discharge means, for example. The method will be described with reference to FIGS. 4A and 4B.

FIG. 4A is a schematic diagram showing an example of a particle production device capable of imparting a flow of a poor solvent to a discharge unit of liquid droplet discharge means.

A particle production device of FIG. 4A includes liquid droplet discharge means 2, a poor solvent accommodation container 61, a stirring member 50, and a pump 31.

The poor solvent accommodation container 61 has a circulation path capable of circulating a liquid and includes a tank 30 as a part of the poor solvent accommodation container 61 in the midway of the circulation path.

FIG. 4B is an enlarged view of the vicinity (broken line portion) of the liquid droplet discharge means 2 in FIG. 4A.

The poor solvent 62 charged into the tank 30 circulates in the poor solvent accommodation container 61 through the liquid droplet discharge means 2 using the pump 31. At this time, a liquid is discharged into the poor solvent 62 from discharge holes of the liquid droplet discharge means 2. Coalescence of particles formed by liquid droplets 21 is suppressed by imparting a flow to the liquid which is the poor solvent 62.

The tank 30 includes the stirring member 50 having a stirring blade, and coalescence of particles can be further suppressed by stirring the liquid, which is the poor solvent 62, with the stirring blade.

In addition, the tank 30 has a heating unit 33 for removing a good solvent contained in the poor solvent 62. The heating with the heating unit 33 is performed within a range where physiological activity of a physiologically active substance contained in particles does not change. In addition, the heating means may not be provided.

Next, another example of the particle production device will be described.

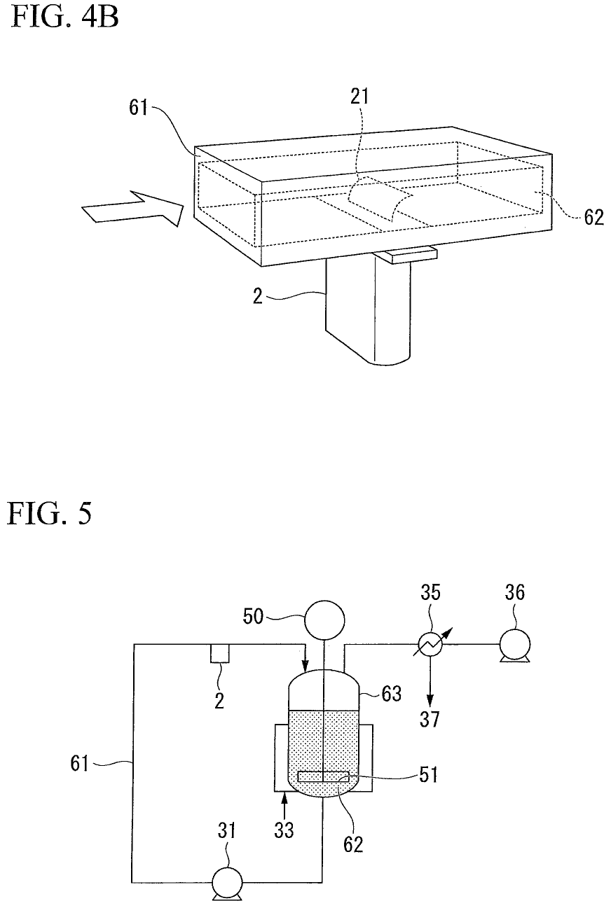

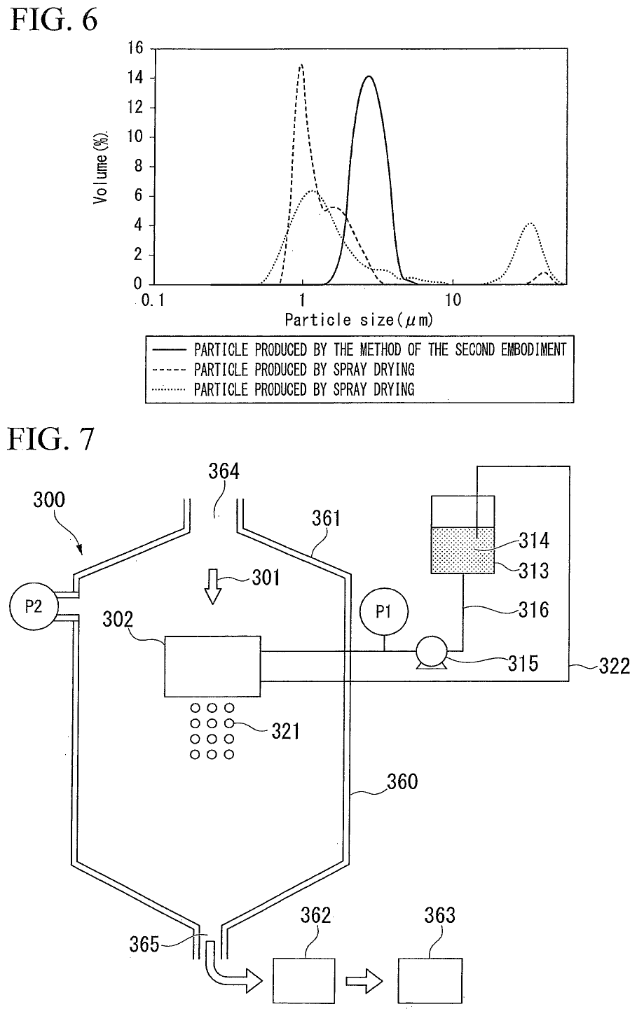

In a case where the amount of good solvent in a poor solvent increases, coalescence of particles increases and the particles easily become coarse. As a method for preventing this, it is preferable to remove the good solvent from the poor solvent to keep the amount of good solvent in the poor solvent small. This method will be described with reference to FIG. 5.

FIG. 5 is a schematic diagram showing an example of a particle production device having good solvent removal means that removes a good solvent.

The particle production device shown in FIG. 5 includes liquid droplet discharge means 2, a poor solvent accommodation container 61, a stirring member 50, a pump 31, a heating unit 33 as good solvent removal means, and a decompression unit 36 (vacuum pump).

The configuration in the vicinity of the liquid droplet discharge means 2 is the same as that of FIGS. 4A and 4B.

The poor solvent accommodation container 61 has a circulation path capable of circulating a liquid and includes a tank 63 as a part of the poor solvent accommodation container 61 in the midway of the circulation path.

The poor solvent 62 charged into the tank 63 circulates in the poor solvent accommodation container 61 through the liquid droplet discharge means 2 using the pump 31. At this time, liquid droplets are discharged into the poor solvent 62 from discharge holes of the liquid droplet discharge means 2. Coalescence of particles formed by liquid droplets 21 is suppressed by imparting a flow to the poor solvent 62.

Furthermore, since the tank 63 is provided with the heating unit 33 and the decompression unit 36, it is possible to remove a good solvent contained in the poor solvent 62. For example, a liquid is decompressed by the decompression unit 36 while the poor solvent is heated by the heating unit 33. Then, the good solvent having a lower boiling point than that of the poor solvent evaporates. The evaporated good solvent is condensed by a condenser 35 and is recovered through a recovery pipe 37. However, the heating with the heating unit 33 is performed within a range where physiological activity of a physiologically active substance contained in particles does not change. In addition, the heating means may not be provided.

### <Method for Producing Particles as Second Embodiment>

A method for producing particles as a second embodiment (in-gas drying) includes: discharging liquid droplets containing a base material, a physiologically active substance having physiological activity, and a solvent into a gas; and granulating particles by vaporizing the solvent from the liquid droplets and removing the solvent which has been contained in the liquid droplets, and includes other steps as necessary.

A plurality of methods are known in the related art as a dry-type granulation method, such as the second embodiment, in which particles are granulated in a gas.

Examples thereof include in-gas pulverization methods such as a method for cooling a melt-kneaded product obtained such that a particle material is melt-kneaded and dispersed uniformly and subsequently pulverizing the cooled melt-kneaded product with a pulverizer to obtain pulverized particles of which the particle diameter is reduced, and a method for freeze-drying a liquid containing a particle material and subsequently pulverizing the freeze-dried liquid with a pulverizer to obtain pulverized particles of which the particle diameter is reduced.

Another examples thereof include spray-drying methods such as a method for spraying a liquid containing a particle material into a gas and drying the sprayed liquid to obtain sprayed particles of which the particle diameter is reduced. As spray methods, there is a pressure nozzle type method in which a liquid is sprayed from nozzles by pressurizing the liquid and a disk type method in which a liquid is sent to a disk rotating at high speed and scattered by a centrifugal force.

In the in-gas pulverization method, a facility used for pulverization is simple, but it is difficult to produce particles with a narrow particle size distribution. Furthermore, in the method for pulverizing a melt-kneaded product, in a case where a physiologically active substance having a property such that physiological activity is changed by heating is contained as a material of particles, the physiological activity of the physiologically active substance changes, and as a result, the amount of physiological activity decreases. In addition, in the method for pulverizing a freeze-dried product, in a case where a physiologically active substance having a property such that physiological activity is changed by the cooling is contained as a material of particles, the physiological activity of the physiologically active substance changes, and as a result, the amount of physiological activity decreases. Furthermore, in the in-gas pulverization method, since a large external stress is generated in a pulverization process, in a case where a physiologically active substance having a property such that physiological activity is changed by the external stress is contained as a material of particles, the physiological activity of the physiologically active substance changes, and as a result, the amount of physiological activity decreases.

The spray-drying method can produce particles having a high proportion (physiologically active substance retention ratio) of a physiologically active substance held in particles during producing particles. However, in general, it is difficult to produce particles with a small particle diameter. In the case of the disk-type spraying method, particles with a small particle diameter can be sometimes produced. However, a large-scale facility is required. In addition, in the spray-drying method, liquid droplets easily coalesce in a gas, and it is difficult to produce particles with a narrow particle size distribution. In order to suppress coalescence of liquid droplets in a gas, it is necessary to heat the liquid droplets after spraying so as to immediately dry the liquid droplets. However, in a case where a physiologically active substance having a property such that physiological activity is changed by heating is contained as a material of particles, the physiological activity of the physiologically active substance changes, and as a result, the amount of physiological activity decreases. Furthermore, in the spray-drying method, since a large external stress is generated by spraying with a high shear force, in a case where a physiologically active substance having a property such that physiological activity is changed by the external stress is contained as a material of particles, the physiological activity of the physiologically active substance changes, and as a result, the amount of physiological activity decreases.

Since the method for producing particles as the second embodiment to be described below does not correspond to the above-described in-gas pulverization method and the spray-drying method, even in the case where a physiologically active substance having a property such that physiological activity is changed by heating, cooling, or external stress is contained as a material of particles, the change in physiological activity of the physiologically active substance is suppressed, and as a result, the decrease in amount of physiological activity is also suppressed.

In addition, as will be described below, the method for producing particles as the second embodiment preferably does not contain using means such as shaking or stirring for imparting external stress by which the physiological activity of a physiologically active substance changes, using heating means for controlling the temperature so that physiological activity of a physiologically active substance changes, and using cooling means for controlling the temperature so that physiological activity of a physiologically active substance changes.

### —Liquid Droplet Discharge Step—

The liquid droplet discharge step of the second embodiment is described in detail in the above-described liquid droplet discharge step of the first embodiment except that liquid droplets are discharged into a gas.

The method for discharging a liquid containing a base material, a physiologically active substance, and a solvent through vibration will be described again as an example of the liquid droplet discharge step of the second embodiment. The discharge method through vibration is not particularly limited, but examples thereof include the following methods. Each means will be described below.

(a) Method using volume change means for changing volume of liquid accommodation unit using vibration

(b) Method using constriction generation means that discharges liquid through a plurality of discharge holes provided in liquid accommodation unit while adding vibration to liquid accommodation unit, and makes liquid into liquid droplets from columnar shape through constricted state

(c) Method using nozzle vibration means for vibrating thin film on which nozzle is formed

The volume change means is not particularly limited as long as it is possible to change the volume of a liquid accommodation unit, and can be appropriately selected depending on the purpose. Examples thereof include a piezoelectric element (also sometimes referred to as a “piezo element”) that expands and contracts when voltage is applied.

Examples of constriction generation means include means using a technique disclosed in Japanese Unexamined Patent Application, First Publication No. 2007-199463. In Japanese Unexamined Patent Application, First Publication No. 2007-199463, means that discharges a liquid from a plurality of nozzle holes provided in a liquid accommodation unit while applying vibration to the liquid accommodation unit using vibration means with a piezoelectric element coming into contact with a part of the liquid accommodation unit, and makes a liquid into liquid droplets from a columnar shape through a constricted state is disclosed.

Examples of nozzle vibration means include means using a technique disclosed in Japanese Unexamined Patent Application, First Publication No. 2008-292976. In Japanese Unexamined Patent Application, First Publication No. 2008-292976, means that discharges a liquid from a plurality of nozzle holes using a thin film, in which a plurality of nozzles provided in a liquid accommodation unit are formed, and a piezoelectric element which is disposed around a region, in which this thin film is deformable, and vibrates the thin film, and makes the liquid into liquid droplets is disclosed.

A piezoelectric element is generally used as means for generating vibration. The piezoelectric element is not particularly limited, and the shape, size, and material thereof can be appropriately selected. For example, it is possible to suitably use a piezoelectric element used for an ink jet discharge method in the related art.

The shape and size of a piezoelectric element is not particularly limited, and can be appropriately selected according to the shape of a discharge hole and the like.

The material of a piezoelectric element is not particularly limited, and can be appropriately selected depending on the purpose. Examples thereof include piezoelectric ceramics such as lead zirconate titanate (PZT), piezoelectric polymers such as polyvinylidene fluoride (PVDF), and single crystals such as crystal, LiNbO3, LiTaO3, and KNbO3.

Discharge holes are not particularly limited, and can be appropriately selected depending on the purpose. Examples thereof include an opening portion provided in a nozzle plate or the like.

The cross-sectional shape and size of a discharge hole can be appropriately selected.

The cross-sectional shape of a discharge hole is not particularly limited, and can be appropriately selected depending on the purpose. Examples thereof include (1) a tapered shape in which the opening diameter decreases from the inside (liquid accommodation unit side) to the outside (a side from which a liquid is discharged), (2) a shape in which the opening diameter decreases from the inside (liquid accommodation unit side) to the outside (a side from which a liquid is discharged) while having a round shape, (3) a shape in which the opening diameter decreases from the inside (liquid accommodation unit side) to the outside (a side from which a liquid is discharged) while having a constant nozzle angle, and (4) a combination of the shape of (1) and the shape of (2). Among these, the shape of (3) is preferable from the viewpoint that pressure applied to a liquid in discharge holes becomes maximum.

The nozzle angle of the shape of (3) is not particularly limited, and can be appropriately selected depending on the purpose. The nozzle angle thereof is preferably 60° to 90°. In the case where the nozzle angle is 60° to 90°, discharge of liquid droplets can be stabilized.

The size of a discharge hole is not particularly limited, and can be appropriately selected depending on the purpose. For example, the diameter thereof is preferably less than 1,000 μm, more preferably greater than or equal to 1.0 μm and less than 1,000 μm, still more preferably 1.0 μm to 500 μm, and particularly preferably 1.0 μm to 50 μm. In a case where the shape of a discharge hole is not a perfect circle, a diameter of a perfect circle having the same area as that of the discharge hole is employed.

A particle composition liquid contains a base material, a physiologically active substance having physiological activity, and a solvent. Since various materials similar to those of the base material and the physiologically active substance contained in particles can be used as the base material and the physiologically active substance contained in the liquid, description thereof will not be repeated, and only the solvent will be described.

### —Solvent—

Examples of solvents include water, aliphatic halogenated hydrocarbons (for example, dichloromethane, dichloroethane, and chloroform), alcohols (for example, methanol, ethanol, and propanol), ketones (for example, acetone and methyl ethyl ketone), ethers (for example, diethyl ether, dibutyl ether, and 1,4-dioxane), aliphatic hydrocarbons (for example, n-hexane, cyclohexane, and n-heptane), aromatic hydrocarbons (for example, benzene, toluene, and xylene), organic acids (for example, acetic acid and propionic acid), esters (for example, ethyl acetate), amides (for example, dimethylformamide and dimethylacetamide), or mixed solvents thereof. These may be used alone or in combination of two or more thereof. Among these, from the viewpoint of solubility, aliphatic halogenated hydrocarbons, alcohols, or mixed solvents thereof are preferable, and dichloromethane, 1,4-dioxane, methanol, ethanol, or mixed solvents thereof are more preferable.

The content of solvent with respect to the mass of the particle composition liquid is preferably 70 mass % to 99.5 mass % and more preferably 90 mass % to 99 mass %. In the case where the content thereof is 70 mass % to 99.5 mass %, the production stability is improved from the viewpoints of liquid viscosity and solubility of a particle material.

The viscosity of a particle composition liquid is not particularly limited, and can be appropriately selected depending on the purpose. The viscosity thereof is preferably 0.5 mPa·s to 15.0 mPa·s and more preferably 0.5 mPa·s to 10.0 mPa·s. The viscosity can be measured, for example, with a viscoelasticity measurement device (device name: MCR rheometer manufactured by AntonPaar) under the conditions of 25° C. and a shear rate of 10 s⁻¹. In the case where the viscosity of a liquid is 0.5 mPa·s to 15.0 mPa·s, discharge can be suitably performed by the above-described means for discharging liquid droplets, which is preferable.

The surface tension of a particle composition liquid is not particularly limited, and can be appropriately selected depending on the purpose. The surface tension thereof is preferably 10 mN/m to 60 mN/m and more preferably 20 mN/m to 50 mN/m. The surface tension can be measured, for example, with a handy surface tensiometer (device name: PocketDyne manufactured by KRUSS) through a maximum bubble pressure method under the conditions of 25° C. and a lifetime of 1,000 ms. In the case where the surface tension of a liquid is 0.5 mPa·s to 15.0 mPa·s, discharge can be suitably performed by the above-described means for discharging liquid droplets, which is preferable.

### —Granulation Step—

The granulation step of the second embodiment is granulating particles by vaporizing a solvent from liquid droplets and removing the solvent which has been contained in the liquid droplets. The granulation step is performed in a gas, and specifically, is preferably performed when liquid droplets discharged into a gas in a liquid droplet discharge step are flying in a gas. By granulating particles through this step, it is possible to make the form of the particles into a solid dispersion and specifically to produce particles in a form in which a physiologically active substance is dispersed in a base material.

Unlike the spray-drying method in the related art, drying with heating or cooling is not required for particles produced through this method. Therefore, this method is particularly advantageous in formation of particles containing a physiologically active substance of which the physiological activity is easily changed by heating or cooling. In addition, it is possible to granulate particles by discharging liquid droplets having a substantially uniform size while controlling the liquid droplets so as not to coalesce, and vaporizing the solvent from the liquid droplets. Therefore, it is possible to produce a large amount of particles having a uniform size, thereby narrowing a particle size distribution. In addition, the particle diameter of particles can also be adjusted by appropriately adjusting the size or the like of discharge holes of discharge means that forms liquid droplets. In a case where discharge means or the like for forming liquid droplets through vibration or the like is used instead of using a pulverization device in which a large external stress is generated or a spray device with a high shear force as means for reducing the particle diameter of particles, even in the case where a physiologically active substance having a property such that physiological activity is changed by external stress is contained as a material of the particles, the change in physiological activity of the physiologically active substance is suppressed, and as a result, the decrease in amount of physiological activity can be suppressed. In addition, in this step, it is unnecessary to bring liquid droplets into contact with a solvent such as water during granulation. Therefore, it is possible to produce particles having a high proportion (physiologically active substance retention ratio) of a physiologically active substance held in particles during producing particles. It is possible to increase the physiological activity ratio of the particles through this step compared to other methods, for example, it is possible to make the physiological activity ratio greater than or equal to 50%.

In the granulation step, particles may be granulated by discharging liquid droplets in conveyance airflow and vaporizing a solvent from the liquid droplets. The method for vaporizing a solvent from liquid droplets using conveyance airflow is not particularly limited, and can be appropriately selected depending on the purpose. For example, a method in which a conveyance direction of conveyance airflow is regarded as a direction substantially vertical to a direction in which liquid droplets are discharged is preferable. In addition, it is preferable that, in the conveyance airflow, the temperature, vapor pressure, the type of gas, and the like be appropriately adjusted. Although heating means may be provided to adjust the temperature of conveyance airflow, discharge is performed while suppressing coalescence of liquid droplets in the granulation step. For this reason, the degree of heating through heating means can be suppressed, and specifically, heating can be applied to such a degree that physiological activity of a physiologically active substance does not change.

In addition, in a case where collected particles maintain a solid state, a solvent may not be completely vaporized, and a drying step may be additionally provided as another step after the collection. In addition, a method for vaporizing a solvent from liquid droplets through application of temperature change, chemical change, or the like may be used.

—In Case of Granulation from Liquid Droplets Containing at Least Two Base Materials—

In a case of producing particles in a form which contains at least two base materials and in which one of the at least two base materials is unevenly distributed on a surface side, it is possible to form particles in the form in a granulation step by appropriately selecting the type of base material to be contained in a particle composition liquid.

In the granulation step, in order to form particles in the form in which one of at least two base materials is unevenly distributed on a surface side, the sizes of contact angles of the at least two base materials are made different from each other. Accordingly, in a case where a solvent is vaporized from liquid droplets in the granulation step, an interaction between base materials becomes large. At this time, since the sizes of the contact angles of the at least two base materials are different from each other, phases of the base materials are easily separated from each other. Then, one of the at least two base materials is unevenly distributed on the surface side. Thereafter, if vaporization of the solvent progresses, particles solidified in this state are formed. According to this method, it is possible to form particles in the form in which one of the at least two base materials is unevenly distributed on the surface side in only one step.

The difference in contact angle between base materials is not particularly limited, and can be appropriately selected depending on the purpose. The difference is preferably greater than or equal to 1.0° and more preferably greater than or equal to 10.0°. In a case where the difference in contact angle between base materials is within the preferred range, phases of the base material are easily separated from each other.

The method for measuring contact angles of base materials is not particularly limited, and can be appropriately selected depending on the purpose. Examples thereof include a measurement method using a contact angle meter. Examples of contact angle meters include a portable contact angle meter PG-X+ which is a mobile contact angle meter manufactured by FIBRO system.

A method for checking the presence or absence of phase separation in at least two base materials is not particularly limited, and can be appropriately selected depending on the purpose. Examples thereof include a method for making a solution, obtained by dissolving at least two base materials in a good solvent, into a thin film shape with a bar coater to check states before, during, and after drying with an optical microscope. Examples of optical microscopes include OLYMPUS BX51 manufactured by Olympus Corporation.

In addition, in a case where a solvent used together with base materials is lipophilic, a base material having a larger contact angle than that of the other base material unevenly distributed inside particles is selected as one base material which unevenly distributed on a surface side. Then, the one base material having a large contact angle has a greater affinity with the solvent than the other base material unevenly distributed inside the particles, and therefore, is easily unevenly distributed on the solvent side, that is, on the surface side.

In addition, in a case where a solvent used together with base materials is hydrophilic, a base material having a smaller contact angle than that of the other base material unevenly distributed inside particles is selected as one base material which unevenly distributed on a surface side. Then, the one base material having a small contact angle has a lower affinity with the solvent than the other base material unevenly distributed inside the particles, and therefore, is easily unevenly distributed on the solvent side, that is, on the surface side.

For this reason, in a case where, for example, it is desired to prepare produce particles that contain a water-soluble, physiologically active substance, as many pharmaceutical compositions do, it is possible to employ a structure in which the physiologically active substance is unevenly distributed inside the particles using a lipophilic solvent, and the inside of the particles is coated with one base material unevenly distributed on a surface side.

In addition, in a case where it is desired to produce particles containing an oil-soluble, physiologically active substance, it is possible to employ a structure in which the physiologically active substance is unevenly distributed inside the particles using a hydrophilic solvent, and the inside of the particles is coated with one base material unevenly distributed on a surface side.

In a case of producing particles that can be incorporated in an enteric pharmaceutical composition using a pH-responsive material as one base material unevenly distributed on a surface side, the pH-responsive material is preferably at least one selected from a cellulosic polymer and a methacrylic acid-based polymer. This is because a cellulosic polymer and a methacrylic acid-based polymer have a larger contact angle than other base materials.

In addition, among cellulosic polymers, at least one selected from hydroxypropyl methylcellulose acetate succinate and hydroxypropyl methylcellulose phthalate is preferable. This is because these have a larger contact angle than other base materials.

In addition, among methacrylic acid-based polymers, an ammonioalkyl methacrylic ester copolymer is preferable. This is because it has a larger contact angle than other base materials.

As a combination of one base material unevenly distributed on a surface side and the other base material unevenly distributed on the inside, a combination of any one selected from poly(meth)acrylic acids, polyglycolic acid, and hydroxypropyl methylcellulose and any one selected from hydroxypropyl cellulose, polyethylene pyrrolidone, and polyalkylene glycols are preferable, for example. This is because these combinations are incompatible with each other and undergo phase separation.

### —Other Steps—

Other steps are not particularly limited, and can be appropriately selected depending on the purpose. Examples thereof include collecting particles.

The collecting produced particles can be suitably carried out with particle collection means. Particle collection means is not particularly limited, and can be appropriately selected depending on the purpose. Examples thereof include a cyclone collector and a bag filter.

### <Particle Production Device as Second Embodiment>

A particle production device as a second embodiment (in-gas drying) includes: liquid droplet discharge means for discharging liquid droplets containing a base material, a physiologically active substance having physiological activity, and a solvent into a gas; and granulation means for granulating particles by vaporizing the solvent from the liquid droplets and removing the solvent which has been contained in the liquid droplets, and includes other means as necessary.

### —Liquid Accommodation Container—

A liquid accommodation container is a container in which a liquid containing a base material, a physiologically active substance, and a solvent is accommodated.

The liquid accommodation container may or may not be flexible. The material of the liquid accommodation container is not particularly limited, and can be appropriately selected depending on the purpose. For example, the material may be made of resin or metal. The structure of the liquid accommodation container is not particularly limited, and can be appropriately selected depending on the purpose. For example, the structure may be a closed structure or may be a non-closed structure.

### —Liquid Droplet Discharge Means—

The liquid droplet discharge means is means for discharging a liquid containing a base material, a physiologically active substance, and a solvent into a gas to form liquid droplets. The liquid droplet formation means is described in detail in the section of the liquid droplet discharge means used for the above-described particle production device as the first embodiment. In a preferred aspect, the liquid droplet discharge means forms liquid droplets by discharging a particle composition liquid through vibration.

The liquid droplet discharge means is connected to the liquid accommodation container. Means for connecting the liquid droplet discharge means to the liquid accommodation container is not particularly limited as long as a liquid can be supplied from the liquid accommodation container to the liquid droplet discharge means, and can be appropriately selected depending on the purpose. Examples thereof include a pipe (such as a tube).

The liquid droplet discharge means preferably has a vibration-applying member that discharges liquid droplets by applying vibration to a liquid. The vibration is not particularly limited, and can be appropriately selected depending on the purpose. For example, the frequency is preferably greater than or equal to 1 kHz, more preferably greater than or equal to 150 kHz, and still more preferably 300 kHz to 500 kHz. It is possible to reproducibly make a liquid column injected from discharge holes into liquid droplets in a case where the vibration is greater than or equal to 1 kHz, and it is possible to improve the production efficiency in a case where the vibration is greater than or equal to 150 kHz.

Examples of the liquid droplet discharge means having a vibration-applying member include an ink jet nozzle. A liquid column resonance method, a film vibration method, a liquid vibration method, and a Rayleigh division method can be used as discharge mechanism of an ink jet nozzle.

### —Granulation Means—

The granulation means is means for granulating particles by vaporizing a solvent from liquid droplets and removing the solvent which has been contained in the liquid droplets.

Examples of granulation means include a member that forms a space for vaporizing a solvent from liquid droplets.

The granulation means preferably has conveyance airflow formation means that forms conveyance airflow.

### Specific Example of Second Embodiment

Next, a specific example of the second embodiment will be described with reference to FIGS. 7 to 9.

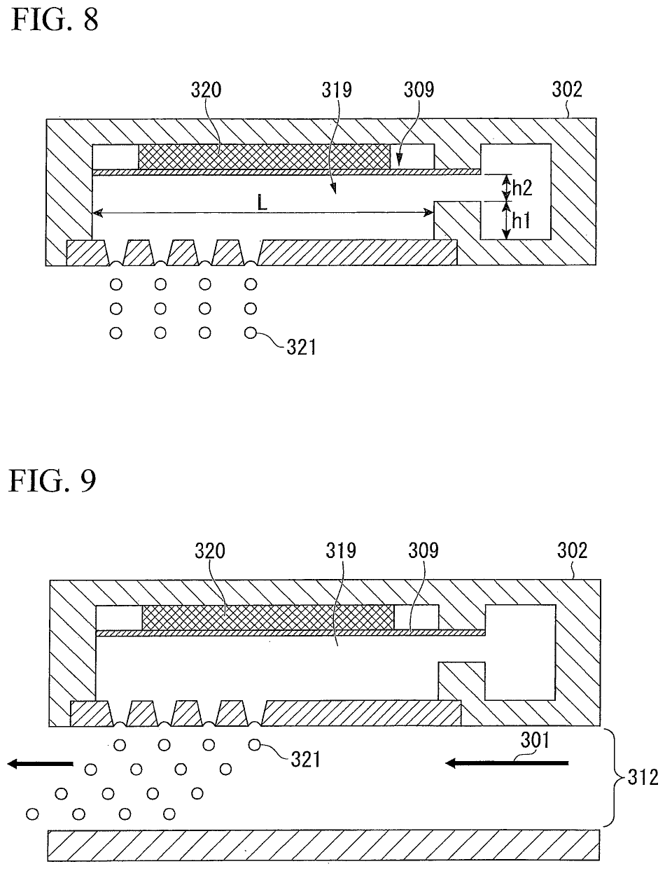

FIG. 7 is a schematic diagram showing an example of a particle production device. FIG. 8 is a schematic cross-sectional view showing an example of liquid droplet discharge means used in a particle production device. FIG. 9 is a schematic cross-sectional view showing another example of liquid droplet discharge means used in a particle production device.

A particle production device 300 shown in FIG. 7 includes liquid droplet discharge means 302, a dry collection unit 360, a conveyance airflow discharge port 365, and a particle storage unit 363. The liquid accommodation container 313 accommodating a liquid 314 and a liquid circulation pump 315, which supplies the liquid 314 accommodated in the liquid accommodation container 313 to the liquid droplet discharge means 302 through a liquid supply pipe 316 and pressure-feeds the liquid 314 in the liquid supply pipe 316 to return the liquid to the liquid accommodation container 313 through a liquid return pipe 322, are connected to the liquid droplet discharge means 302, and the liquid 314 can be supplied to the liquid droplet discharge means 302 at all times. Pressure measurement devices P1 and P2 are respectively provided in the liquid supply pipe 316 and the dry collection unit. The liquid feed pressure to the liquid droplet discharge means 302 and the pressure in the dry collection unit are managed by the pressure measurement devices P1 and P2. At this time, in a case where a pressure measurement value of P1 is larger than that of P2, there is a concern that the liquid 314 may ooze out from discharge holes. In a case where a pressure measurement value of P1 is smaller than that of P2, there is a concern that discharge may stop due to introduction of a gas into the liquid droplet discharge means 302. Therefore, it is preferable that the pressure measurement values of P1 and P2 be the same as each other.

Descending airflow (conveyance airflow) 301 formed from a conveyance airflow introduction port 364 is formed in a chamber 361. Liquid droplets 321 discharged from the liquid droplet discharge means 302 are conveyed downward not only by gravity but also by the conveyance airflow 301, collected by particle collection means 362 through the conveyance airflow discharge port 365, and stored in the particle storage unit 363.

In a liquid droplet discharge step, if discharged liquid droplets come into contact with each other before drying, the liquid droplets sometimes coalesce. In order to obtain particles having a narrow particle size distribution, the distance between discharged liquid droplets is preferably maintained. However, discharged liquid droplets have a constant initial velocity and eventually stall due to air resistance. In a case where liquid droplets discharged later catch up with the stalled liquid droplets and drying of the liquid droplets is insufficient, the liquid droplets can coalesce. In order to prevent the coalescence, it is preferable to convey the liquid droplets while drying the liquid droplets while suppressing the coalescence using the conveyance airflow 301 so as to suppress the decrease in velocity of the liquid droplets and prevent the liquid droplets from coming into contact with each other. For this reason, the conveyance airflow 301 is preferably arranged in the same direction as the liquid droplet discharge direction in the vicinity of the liquid droplet discharge means 302. Even if liquid droplets come into contact with each other, these do not coalesce if these are sufficiently dried by the time of contact. Therefore, the conveyance airflow 301 may not be used in such a case.

FIG. 8 is an enlarged view of the liquid droplet discharge means of the particle production device of FIG. 7. As shown in FIG. 8, the liquid droplet discharge means 302 has volume change means 320, an elastic plate 309, and a liquid accommodation unit 319. The liquid droplet discharge means 302 deforms when voltage is applied to the volume change means 320 to reduce the volume of the liquid accommodation unit 319. Accordingly, a liquid stored in the liquid accommodation unit 319 is discharged as the liquid droplets 321 from discharge holes.

FIG. 9 is a view showing another aspect of liquid droplet discharge means of a particle production device. As shown in FIG. 9, conveyance airflow 301 may be in a direction substantially perpendicular to a discharge direction in a airflow passage 312. The conveyance airflow 301 may have an angle, and preferably has an angle so that liquid droplets are separated from liquid droplet discharge means 302. In a case where the volume of a liquid accommodation unit 319 is changed by an elastic plate 309 due to volume change means 320 to discharge liquid droplets 321, and the conveyance airflow 301 for preventing coalescence is applied to the discharged liquid droplets 321 in the direction substantially perpendicular to the discharged liquid droplets, discharge holes are preferably arranged as shown in FIG. 9 so that trajectories, on which the liquid droplets 321 pass when being conveyed by the conveyance airflow 301 for preventing coalescence from the discharge holes, do not overlap each other.

After the coalescence is prevented by the conveyance airflow 301, particles may be carried to particle collection means using another airflow.

The velocity of the conveyance airflow is preferably the same as or greater than or equal to the liquid droplet discharge rate. In a case where the velocity of conveyance airflow is higher than the liquid droplet discharge rate, the coalescence of the liquid droplets can be suppressed. In addition, a chemical substance promoting drying of the liquid droplets may be mixed in the conveyance airflow. The airflow state of the conveyance airflow is not limited, and may be laminar flow, swirling flow, or turbulent flow. The type of gas constituting the conveyance airflow is not particularly limited, and can be appropriately selected depending on the purpose. Air may be used, or a nonflammable gas such as nitrogen may be used. In addition, the temperature of the conveyance airflow can be appropriately adjusted, but is a temperature at which the physiological activity of a physiologically active substance contained in liquid droplets is not changed by the temperature of the airflow.

In a case where the amount of remaining solvent contained in particles obtained by the particle collection means 362 shown in FIG. 7 is large, it is preferable to perform secondary drying as necessary in order to reduce the amount of remaining solvent. Generally known drying means such as fluidized-bed drying or vacuum drying can be used as the secondary drying.

### EXAMPLES

Hereinafter, production examples of particles will be described, but the present invention is not limited to these production examples.

### <Preparation Example of Particle Material-Mixed Solution 1A>

HRP (peroxidase derived from horseradish, product code PEO-131, manufactured by TOYOBO Co., LTD.) as a physiologically active substance was added to pure water as a solvent to adjust an aqueous solution having an HRP content of 2.00 mg/mL. In addition, HPC (hydroxypropyl cellulose 2.0 to 2.9, product code 082-07925, manufactured by Wako Pure Chemical Industries, Ltd.) as a base material was added to pure water as a solvent to adjust an aqueous solution having a hydroxypropyl cellulose content of 10.89 mg/mL.

Next, the aqueous solution of HRP and the aqueous solution of HPC were mixed with each other so that the mass ratio became 1:9, and the mixed solution was gently stirred manually. The solid content of this mixed solution was 1 mass %, and the content of the physiologically active substance was 200 μg/mL.

Thereafter, the obtained mixed solution was passed through a filter (CES-005-M47DK) having an average pore diameter of 0.45 μm to obtain a particle material-mixed solution 1A.

### Production Example of Particles 1A-1 (Liquid Column Resonance)

The obtained particle material-mixed solution 1A was discharged from a discharge port and made into liquid droplets using liquid droplet discharge means in which the number of openings of the discharge port in FIG. 8 was set to one per liquid column resonance liquid chamber, and a solvent was removed from the liquid droplets using the particle production device of FIG. 9 to obtain particles 1A-1.

The volume-average particle diameter (Dv) of the obtained particles 1A-1 was 2.71 μm, the number-average particle diameter (Dn) thereof was 2.37 μm, and the particle size distribution (Dv/Dn) thereof was 1.14. These were measured with a laser diffraction-scattering-type particle size distribution measuring device (device name: Microtrack MT30001 manufactured by MicrotracBEL Corp.)

In addition, D90, D50, and D10 of the obtained particles 1A-1 were respectively 3.52 μm, 2.64 μm, and 2.18 μm, and the particle size distribution (R.S.F) thereof was 0.51. These were measured with a laser diffraction-scattering-type particle size distribution measuring device (device name: Microtrack MT30001 manufactured by MicrotracBEL Corp.)

The particle production conditions were as follows.

### —Particle Production Conditions—

Shape of discharge port: perfect circle
Diameter of discharge port: 8.0 μm
Temperature of liquid droplet discharge means: 40° C.
Dry airflow rate: dry nitrogen of 50 L/minute

### Production Example of Particles 1A-2 (Spray Drying)

The obtained particle material-mixed solution 1A was discharged using spray-drying means (4-fluid nozzle manufactured by Fujisaki Electric Co., Ltd.) to obtain particles 1A-2.

The spray-drying conditions were as follows.

### —Spray-Drying Conditions—

Feeding amount of particle material-mixed solution 1A to nozzle: 10 mL/minute
Dry airflow rate: dry nitrogen of 30 L/min
Orifice pressure: 1.3 kPa
Temperature (inlet): 75° C.
Temperature (outlet): 30° C. to 35° C.

### Production Example of Particles 1A-3 (Freeze Drying)

The obtained particle material-mixed solution 1A was freeze-dried with a freeze-dryer (FDU-2110 type freeze-drying unit and DRC-1000 type dry chamber, both manufactured by Tokyo Rikakikai Co., Ltd.), and the dried product was pulverized to obtain particles 1A-3.

The freeze-drying conditions were as follows.

### —Freeze-Drying Conditions—

Pre-freezing: −40° C. for 4 to 6 hours
Primary freezing: −10° C. for 5 hours
Secondary freezing: 20° C. for 12 hours

### [Quantification of Content of Physiologically Active Substance (HRP)]

A calibration curve showing a relationship between the concentration of HRP (peroxidase derived from horseradish, PEO-131, manufactured by TOYOBO Co., LTD.) dissolved in pure water and the absorbance at a measurement wavelength of 400 nm was created. A microsample spectrophotometer (SimpliNano manufactured by GE healthcare) was used for measuring the absorbance.

Next, the absorbances of the produced particles 1A-1 to 1A-3 at a measurement wavelength of 400 nm were measured, and the content of HRP of each particle group was calculated based on the created calibration curve. The results are shown in Table 1 below. The numerical values of the contents shown in Table 1 indicate numerical values when a case (theoretical value) where the total amount of HRP in the particle material-mixed solution 1A is contained in produced particles is regarded as 100%.

### [Quantification of Activity of Physiologically Active Substance (HRP)]

Activity of HRP in the particles 1A-1 to 1A-3 was evaluated according to the protocol of a kit (Pierce TMB Substrate Kit, manufactured by Thermo Fisher Scientific Inc., product code 34021). The results are shown in Table 1 below. The numerical values of the activity shown in Table 1 indicate numerical values when a case (theoretical value) where the total amount of HRP in the particle material-mixed solution 1A has activity in produced particles is regarded as 100%.

Devices and reagents used were as follows.

### —Devices Used—

Absorption microplate reader Multiscan GO (manufactured by Thermo Fisher Scientific Inc.)
Nunc Edge 2.0 96-Well Plates (manufactured by Thermo Fisher Scientific Inc., product code 167425)

### —Reagents Used—

HRP (peroxidase derived from horseradish, product code PEO-131, manufactured by TOYOBO Co., LTD.)
HPC (hydroxypropyl cellulose 2.0 to 2.9, product code 082-07925, manufactured by Wako Pure Chemical Industries, Ltd.)
Pierce TMB Substrate Kit (manufactured by Thermo Fisher Scientific Inc., product code 34021)

TABLE 1

| HRP manufacturer | Particles | Production method | Content (%) | Activity (%) |
|---|---|---|---|---|
| TOYOBO Co., LTD. | Particles 1A-1 | Liquid column | 97 | 99 |
| | | resonance | | |
| | Particles 1A-2 | Spray drying | 106 | 103 |
| | Particles 1A-3 | Freeze drying | 109 | 99 |

According to the results shown in Table 1, the content and activity of the physiologically active substance in the particles 1A-1 are shown as approximately theoretical values. This shows that the results are superior when expensive substances such as proteins are selected as physiologically active substances.

### Preparation Example of Particle Material-Mixed Solution 2A

A particle material-mixed solution 2A was obtained through the same method as that in the preparation example of the particle material-mixed solution 1A except that catalase (derived from bovine liver, product code 039-12901, manufactured by Wako Pure Chemical Industries, Ltd.) was used as a physiologically active substance instead of HRP (peroxidase derived from horseradish, product code PEO-131, manufactured by TOYOBO Co., LTD.) in the preparation example of the particle material-mixed solution 1A.

### Production Example of Particles 2A-1 (Liquid Column Resonance)

Particles 2A-1 were obtained through the same method as in the production example of the particles 1A-1 except that the particle material-mixed solution 2A was used instead of the particle material-mixed solution 1A in the production example of the particles 1A-1.

### Production Example of Particles 2A-2 (Spray Drying)

Particles 2A-2 were obtained through the same method as in the production example of the particles 1A-2 except that the particle material-mixed solution 2A was used instead of the particle material-mixed solution 1A in the production example of the particles 1A-2.

### Production Example of Particles 2A-3 (Freeze Drying)

Particles 2A-3 were obtained through the same method as in the production example of the particles 1A-3 except that the particle material-mixed solution 2A was used instead of the particle material-mixed solution 1A in the production example of the particles 1A-3.

### Preparation Example of Particle Material-Mixed Solution 2B

A particle material-mixed solution 2B was obtained through the same method as that in the preparation example of the particle material-mixed solution 1A except that catalase (derived from bovine liver, product code C9322, manufactured by SIGMA Corporation) was used as a physiologically active substance instead of HRP (peroxidase derived from horseradish, product code PEO-131, manufactured by TOYOBO Co., LTD.) in the preparation example of the particle material-mixed solution 1A. The catalase (derived from bovine liver, product code C9322, manufactured by SIGMA Corporation) was used after removing a stabilizer (trehalose) through dialysis in advance. The dialysis conditions were as follows.

### —Dialysis Conditions—

Filter: Slide-A-Lyzer MINI Dialysis Device, 10K MWCO
Buffer: 10 mM sodium citrate aqueous solution
Tube used: 1.5 mL micro-tube
Buffer amount: 1.1 mL
Charge amount: 100 μL at 5 mg/mL
Centrifugation: 200 rpm for 60 minutes, performed 4 times

### Production Example of Particles 2B-1 (Liquid Column Resonance)

Particles 2B-1 were obtained through the same method as in the production example of the particles 1A-1 except that the particle material-mixed solution 2B was used instead of the particle material-mixed solution 1A in the production example of the particles 1A-1.

### Production Example of Particles 2B-2 (Spray Drying)

Particles 2B-2 were obtained through the same method as in the production example of the particles 1A-2 except that the particle material-mixed solution 2B was used instead of the particle material-mixed solution 1A in the production example of the particles 1A-2.

### Production Example of Particles 2B-3 (Freeze Drying)

Particles 2B-3 were obtained through the same method as in the production example of the particles 1A-3 except that the particle material-mixed solution 2B was used instead of the particle material-mixed solution 1A in the production example of the particles 1A-3.

### [Quantification of Activity of Physiologically Active Substance (Catalase)]

Activity of catalase in the particles 2A-1 to 2A-3 and 2B-1 to 2B-3 was evaluated according to the protocol of a kit (Catalase Colorimetric Activity Kit, manufactured by Invitrogen, product code EIACATC). The results are shown in Table 2 below. The numerical values of the activity shown in Table 2 indicate numerical values when a case (theoretical value) where the total amount of catalase in the particle material-mixed solution 2A or 2B has activity in produced particles is regarded as 100%.

Devices and reagents used were as follows.

### —Devices Used—

Absorption microplate reader Multiscan GO (manufactured by Thermo Fisher Scientific Inc.)
Nunc Edge 2.0 96-Well Plates (manufactured by Thermo Fisher Scientific Inc., product code 167425)

### —Reagents Used (in Case of Quantitatively Determining Activity of Particles 2A-1 to 2A-3)—

Catalase (derived from bovine liver, product code 039-12901, manufactured by Wako Pure Chemical Industries, Ltd.)
HPC (hydroxypropyl cellulose 2.0 to 2.9, product code 082-07925, manufactured by Wako Pure Chemical Industries, Ltd.)
Catalase Colorimetric Activity Kit (manufactured by Invitrogen, product code EIACATC)

### —Reagents Used (in Case of Quantitatively Determining Activity of Particles 2B-1 to 2B-3)—

Catalase (derived from Bovine liver, product code C9322, manufactured by SIGMA Corporation)
HPC (hydroxypropyl cellulose 2.0 to 2.9, product code 082-07925, manufactured by Wako Pure Chemical Industries, Ltd.)
Catalase Colorimetric Activity Kit (manufactured by Invitrogen, product code EIACATC)

TABLE 2

| Catalase manufacturer | Particles | Production Method | Activity (%) |
|---|---|---|---|
| Wako Pure Chemical | Particles 2A-1 | Liquid column | 98 |
| Industries, Ltd. | | resonance | |
| | Particles 2A-2 | Spray drying | 95 |
| | Particles 2A-3 | Freeze drying | 92 |
| SIGMA Corporation | Particles 2B-1 | Liquid column | 100 |
| | | resonance | |
| | Particles 2B-2 | Spray drying | 98 |
| | Particles 2B-3 | Freeze drying | 87 |

According to the results shown in Table 2, the activities of the physiologically active substance in the particles 2A-1 and 2B-1 are shown as approximately theoretical values. This shows that the results are superior when expensive substances such as proteins are selected as physiologically active substances.

### Preparation Example of Particle Material-Mixed Solution 3A

A particle material-mixed solution 3A was obtained through the same method as that in the preparation example of the particle material-mixed solution 1A except that LDH (lactate dehydrogenase derived from rabbit muscle, product code 10127876001, manufactured by Roche) was used as a physiologically active substance instead of HRP (peroxidase derived from horseradish, product code PEO-131, manufactured by TOYOBO Co., LTD.) in the preparation example of the particle material-mixed solution 1A.

The LDH (lactate dehydrogenase derived from rabbit muscle, product code 10127876001, manufactured by Roche) was used after removing ammonium sulfate through dialysis in advance. The dialysis conditions were as follows.

### —Dialysis Conditions—

Filter: Slide-A-Lyzer MINI Dialysis Device, 10K MWCO
Tube used: 1.5 mL micro-tube
Buffer amount: 1.1 mL
Charge amount: 100 μL
Centrifugation: 200 rpm for 60 minutes, performed 4 times

### Production Example of Particles 3A-1 (Liquid Column Resonance)

Particles 3A-1 were obtained through the same method as in the production example of the particles 1A-1 except that the particle material-mixed solution 3A was used instead of the particle material-mixed solution 1A in the production example of the particles 1A-1.

### Production Example of Particles 3A-2 (Spray Drying)

Particles 3A-2 were obtained through the same method as in the production example of the particles 1A-2 except that the particle material-mixed solution 3A was used instead of the particle material-mixed solution 1A in the production example of the particles 1A-2.

### Production Example of Particles 3A-3 (Freeze Drying)

Particles 3A-3 were obtained through the same method as in the production example of the particles 1A-3 except that the particle material-mixed solution 3A was used instead of the particle material-mixed solution 1A in the production example of the particles 1A-3.

### Preparation Example of Particle Material-Mixed Solution 3B

A particle material-mixed solution 3B was obtained through the same method as that in the preparation example of the particle material-mixed solution 1A except that LDH (lactate dehydrogenase derived from rabbit muscle, product code L2500-5KU, manufactured by SIGMA Corporation) was used as a physiologically active substance instead of HRP (peroxidase derived from horseradish, product code PEO-131, manufactured by TOYOBO Co., LTD.) in the preparation example of the particle material-mixed solution 1A.

The LDH (lactate dehydrogenase derived from rabbit muscle, product code L2500-5KU, manufactured by SIGMA Corporation) was used after removing ammonium sulfate through dialysis in advance. The dialysis conditions were as follows.

### —Dialysis Conditions—

Filter: Slide-A-Lyzer MINI Dialysis Device, 10K MWCO
Tube used: 1.5 mL micro-tube
Buffer amount: 1.1 mL
Charge amount: 100 μL
Centrifugation: 200 rpm for 60 minutes, performed 4 times

### Production Example of Particles 3B-1 (Liquid Column Resonance)

Particles 3B-1 were obtained through the same method as in the production example of the particles 1A-1 except that the particle material-mixed solution 3B was used instead of the particle material-mixed solution 1A in the production example of the particles 1A-1.

### Production Example of Particles 3B-2 (Spray Drying)

Particles 3B-2 were obtained through the same method as in the production example of the particles 1A-2 except that the particle material-mixed solution 3B was used instead of the particle material-mixed solution 1A in the production example of the particles 1A-2.

### Production Example of Particles 3B-3 (Freeze Drying)

Particles 3B-3 were obtained through the same method as in the production example of the particles 1A-3 except that the particle material-mixed solution 3B was used instead of the particle material-mixed solution 1A in the production example of the particles 1A-3.

### [Quantification of Activity of Physiologically Active Substance (LDH)]

LDH activity in the particles 3A-1 to 3A-3 and 3B-1 to 3B-3 was evaluated according to the protocol of a kit (Pierce LDH Cytotoxicity Assay Kit, manufactured by Thermo Fisher Scientific Inc., product code 88953). The results are shown in Table 3 below. The numerical values of the activity shown in Table 3 indicate numerical values when a case (theoretical value) where the total amount of LDH in the particle material-mixed solution 3A or 3B has activity in produced particles is regarded as 100%.

Devices and reagents used were as follows.

### —Devices Used—

Absorption microplate reader Multiscan GO (manufactured by Thermo Fisher Scientific Inc.)
Nunc Edge 2.0 96-Well Plates (manufactured by Thermo Fisher Scientific Inc., product code 167425)

### —Reagents Used (in Case of Quantitatively Determining Activity of Particles 3A-1 to 3A-3)—

LDH (lactate dehydrogenase derived from rabbit muscle, product code 10127876001, manufactured by Roche)
HPC (hydroxypropyl cellulose 2.0 to 2.9, product code 082-07925, manufactured by Wako Pure Chemical Industries, Ltd.)
Pierce LDH Cytotoxicity Assay Kit (manufactured by Thermo Fisher Scientific Inc., product code 88953)

### —Reagents Used (in Case of Quantitatively Determining Activity of Particles 3B-1 to 3B-3)—

LDH (lactate dehydrogenase derived from rabbit muscle, product code L2500-5KU, manufactured by Sigma)
HPC (hydroxypropyl cellulose 2.0 to 2.9, product code 082-07925, manufactured by Wako Pure Chemical Industries, Ltd.)
Pierce LDH Cytotoxicity Assay Kit (manufactured by Thermo Fisher Scientific Inc., product code 88953)

TABLE 3

| LDH manufacturer | Particles | Production Method | Activity (%) |
|---|---|---|---|
| Roche | Particles 3A-1 | Liquid column | 95 |
| | | resonance | |
| | Particles 3A-2 | Spray drying | 33 |
| | Particles 3A-3 | Freeze drying | 92 |
| SIGMA Corporation | Particles 3B-1 | Liquid column | 79 |
| | | resonance | |
| | Particles 3B-2 | Spray drying | 13 |
| | Particles 3B-3 | Freeze drying | 17 |

According to the results shown in Table 3, the activity of the physiologically active substance in the particles 3A-1 is shown as approximately theoretical value. This shows that the results are superior when expensive substances such as proteins are selected as physiologically active substances. In addition, the activity of the particles 3A-2 produced through spray drying is low whereas the activity of the physiologically active substance in the particles 3A-1 is shown as approximately theoretical value. This indicates that the results are superior in a case where physiologically active substances, such as proteins, which have a property such that physiological activity is changed by heating or external stress are used.

According to the results shown in Table 3, the physiologically active substance in the particles 3B-1 has high activity. This shows that the results are superior when expensive substances such as proteins are selected as physiologically active substances. In addition, the activity of the particles 3B-2 produced through spray drying and the activity of the particles 3B-3 produced through freeze drying are low whereas the physiologically active substance in the particles 3B-1 has high activity. This indicates that the results are superior in a case where physiologically active substances, such as proteins, which have a property such that physiological activity is changed by heating, cooling, or external stress are used.

According to the results shown in Table 3, the activity of the particles produced through freeze drying differs according to the manufacturer of LDH. It is inferred that this is due to isozymes.

### Preparation Example of Particle Material-Mixed Solution 4A

A particle material-mixed solution 4A was obtained through the same method as that in the preparation example of the particle material-mixed solution 1A except that an anti-rabbit IgG goat antibody (polyclonal, manufactured by SIGMA Corporation, product code R5506) was used as a physiologically active substance instead of HRP (peroxidase derived from horseradish, product code PEO-131, manufactured by TOYOBO Co., LTD.) in the preparation example of the particle material-mixed solution 1A.

### Production Example of Particles 4A-1 (Liquid Column Resonance)

Particles 4A-1 were obtained through the same method as in the production example of the particles 1A-1 except that the particle material-mixed solution 4A was used instead of the particle material-mixed solution 1A in the production example of the particles 1A-1.

### Production Example of Particles 4A-2 (Spray Drying)

Particles 4A-2 were obtained through the same method as in the production example of the particles 1A-2 except that the particle material-mixed solution 4A was used instead of the particle material-mixed solution 1A in the production example of the particles 1A-2.

### Production Example of Particles 4A-3 (Freeze Drying)

Particles 4A-3 were obtained through the same method as in the production example of the particles 1A-3 except that the particle material-mixed solution 4A was used instead of the particle material-mixed solution 1A in the production example of the particles 1A-3.

### [Quantification of Activity of Physiologically Active Substance (Anti-Rabbit IgG Goat Antibody)]

Activity of anti-rabbit IgG goat antibodies in the particles 4A-1 to 4A-3 was evaluated through ELISA (sandwich method). The results are shown in Table 4 below. The numerical values of the activity shown in Table 4 indicate numerical values when a case (theoretical value) where the total amount of anti-rabbit IgG goat antibody in the particle material-mixed solution 4A has activity in produced particles is regarded as 100%.

The ELISA (sandwich method) was carried out according to the standard method, and devices and reagents used were as follows.

### —Devices Used—

Absorption microplate reader Multiscan GO (manufactured by Thermo Fisher Scientific Inc.)
Nunc MaxiSorp flat-bottom (manufactured by Thermo Fisher Scientific Inc., product code 44-2404-21)

### —Reagents Used—

Primary antibody: anti-rabbit IgG goat antibody (polyclonal, manufactured by SIGMA Corporation, product code R5506)
Antigen: IgG derived from rabbit serum (manufactured by SIGMA Corporation, product code 15006)
Secondary antibody: anti-rabbit IgG goat antibody-peroxidase label (polyclonal, manufactured by SIGMA Corporation, product code A0545)
HPC (hydroxypropyl cellulose 2.0 to 2.9, product code 082-07925, manufactured by Wako Pure Chemical Industries, Ltd.)
Bovine serum albumin (BSA) (pH of 7.0, manufactured by Wako Pure Chemical Industries, Ltd., product code 019-23293)
Polyoxyethylene (20) sorbitan monolaurate (manufactured by Wako Pure Chemical Industries, Ltd., product code 166-21213)
Pierce TMB Substrate Kit (manufactured by Thermo Fisher Scientific Inc., product code 34021)

TABLE 4

| Antibody manufacturer | Particles | Production method | Activity (%) |
|---|---|---|---|
| SIGMA Corporation | Particles 4A-1 | Liquid column | 99 |
| | | resonance | |
| | Particles 4A-2 | Spray drying | 100 |
| | Particles 4A-3 | Freeze drying | 102 |

According to the results shown in Table 4, the activity of the physiologically active substance in the particles 4A-1 is shown as approximately theoretical value. This shows that the results are superior when expensive substances such as antibodies are selected as physiologically active substances.

### REFERENCE SIGNS LIST

1 particle production device
2 liquid droplet discharge means
13 liquid accommodation container
61 poor solvent accommodation container
300 particle production device
302 liquid droplet discharge means
313 liquid accommodation container
361 chamber

### CITATION LIST

### Patent Literature

[Patent Literature 1] Japanese Unexamined Patent Application, First Publication No. H11-114027

1: Particles, comprising:
at least one base material; and
a physiologically active substance having physiological activity,
wherein the physiologically active substance is contained and dispersed in the at least one base material, and
wherein the physiologically active substance has a property such that the physiological activity is changed by heating, cooling, or external stress.

2: The particles according to claim 1,
wherein a physiological activity ratio, which is a ratio of an amount of the physiological activity of particles made of materials to an amount of the physiological activity of the materials used for producing the particles, is greater than or equal to 50%.

3: The particles according to claim 1, which are a solid dispersion.

4: The particles according to claim 1,
wherein the at least one base material contains at least one selected from the group consisting of lactic acid-glycolic acid copolymer, polylactic acid, and hydroxypropyl cellulose.

5: The particles according to claim 1,
wherein the at least one base material contains a biodegradable polymer.

6: The particles according to claim 1,
wherein the physiologically active substance contains at least one selected from the group consisting of a protein and a nucleic acid.

7: The particles according to claim 1,
wherein the physiologically active substance contains at least one selected from the group consisting of an antibody and an enzyme.

8: The particles according to claim 1, which have a narrow particle size distribution.

9: The particles according to claim 1, which have a volume-average particle diameter (Dv) of 10 nm to 200 nm.

10: The particles according to claim 1, which have a volume-average particle diameter (Dv) of 1 μm to 100 μm.

11: The particles according to claim 10, which do not substantially contain a surfactant.

12: The particles according to claim 10, comprising:
at least two base materials,
wherein one of the at least two base materials is unevenly distributed on a surface side of the particles.

13: A pharmaceutical composition, comprising:
the particles according to claim 1.

14: A method for producing particles, comprising:
discharging liquid droplets containing a base material, a physiologically active substance having physiological activity, and a solvent; and
granulating particles by removing the solvent from the liquid droplets,
wherein the physiologically active substance has a property such that the physiological activity is changed by heating, cooling, or external stress.

15: The method for producing particles according to claim 14,
wherein the solvent is a good solvent for the base material,
wherein the discharging comprises discharging the liquid droplets into a poor solvent of the base material, and
wherein the granulating comprises bringing the liquid droplets into contact with the poor solvent.

16: The method for producing particles according to claim 14,
wherein the discharging comprises discharging the liquid droplets into a gas through vibration, and
wherein the granulating comprises vaporizing the solvent from the liquid droplets.

17: The method for producing particles according to claim 14,
wherein the external stress resulting in changing the physiological activity of the physiologically active substance is not used for applying external stress.

18: The method for producing particles according to claim 14,
wherein the heating resulting in changing the physiological activity of the physiologically active substance is not used for controlling temperature.

19: The method for producing particles according to claim 14,
wherein the cooling resulting in changing the physiological activity of the physiologically active substance is not used for controlling temperature.
